(19) United States
(12) Patent Application Publication
(10) Pub. No.: US 2004/0009956 A1
(43) Pub. Date: Jan. 15, 2004

(54) Inhibition of protein tyrosine phosphatases and SH2 domains by a neutral phosphotyrosine mimetic

(76) Inventors: Dehua Pei, Columbus, OH (US); Junguk Park, Columbus, OH (US); Hua Fu, Beijing (CN)

Correspondence Address: CALFEE HALTER & GRISWOLD, LLP, 800 SUPERIOR AVENUE, SUITE 1400, CLEVELAND, OH, 44114, US

(21) Appl. No.: 10/425,923

(22) Filed: Apr. 29, 2003

Related U.S. Application Data
(60) Provisional application No. 60/376,386, filed on Apr. 29, 2002.

(51) Int. Cl. A61K 31/66

(52) U.S. Cl. 514/114

(57) ABSTRACT

Methods of inhibiting protein tyrosine phosphatases (PTPs) and Src homology 2 domains (SH2) using neutral phosphotyrosine (pY) mimetics (PTP inhibitors) are provided. Neutral pY mimetics, or PTP inhibitors comprising an aldehyde or mono-ketone substituted aryl group are also provided. Substituents on the aryl groups of the PTP inhibitors provide further affinity for particular PTPs. PTP inhibitors comprising a reactive tripeptide substituent are also provided. Methods of using the neutral, reversible inhibitors as probes for studying the physiological functions of PTPs and SH2 domains are also provided. Methods of treating type II diabetes and obesity by administering a neutral pY mimetic that selectively and reversibly binds with PTP1B are also provided.

### CROSS-REFERENCE TO RELATED APPLICATIONS

This application claims priority to provisional application No. 60/376,386 filed Apr. 29, 2002, the entirety of which is incorporated herein by reference.

### STATEMENT REGARDING FEDERALLY FUNDED RESEARCH

This invention was made, at least in part, with government support under National Institutes of Health Grants AI40575 and GM62820. The U.S. government may have certain rights in this invention.

### BACKGROUND

Protein tyrosine phosphatases (PTPs) are a diverse family of enzymes that catalyze the hydrolysis of phosphotyrosine (pY) residues in proteins. These enzymes, together with the protein tyrosine kinases (PTKs), control the level of intracellular tyrosine phosphorylation, thus regulating many cellular functions (1). The malfunction of either PTKs or PTPs can lead to a variety of human diseases and conditions (2, 3). Therefore, inhibitors against these signaling molecules provide potential therapeutic agents. For example, a great deal of effort is currently being made by both academic and industrial labs to develop specific inhibitors for PTP1B, which has been shown to be a promising target for the treatment of type II diabetes (4) and obesity.

Type II diabetes is now widespread in every industrialized country in the world. More than 14 million Americans have type II diabetes. Doctors think the reason this form of diabetes is so common today is because increasing numbers of people are eating more, exercising less and becoming overweight. Most (but not all) people who have type II diabetes are overweight. As the population ages, more and more people will develop type II diabetes, which usually occurs after age 40.

Each year about 200,000 deaths, 400,000 heart attacks, 130,000 strokes, 60,000 amputations, 10,000 new cases of kidney failure requiring dialysis or transplantation and 6,000 new cases of blindness result from type II diabetes. Type II diabetes also leads to other disabilities, especially nerve damage that often results in erectile dysfunction, numbness and weakness, or intractable nausea, vomiting and diarrhea. The disabilities caused by diabetes result in 200 million days of restricted activity, 100 million days of bed rest, and direct and indirect costs of more than $25 billion in the United States alone.

Furthermore, recently compiled data show that approximately 150 million people have diabetes mellitus worldwide, and that this number may well double by the year 2025. Much of this increase will occur in developing countries and will be due to population growth, ageing, unhealthy diets, obesity and sedentary lifestyles. It is estimated that by 2025, while most people with diabetes in developed countries will be aged 65 years or more, in developing countries most will be in the 45-64 year age bracket and affected in their most productive years.

Diabetes is a serious and costly disease which is becoming increasingly common, especially in developing countries and disadvantaged minorities. As the incidence of type II diabetes, and impaired glucose tolerance increases, the need for effective treatments increases. In addition, treatments for obesity may provide an effective method of prevention for type II diabetes.

In addition to providing therapeutic agents, specific PTK and PTP inhibitors would also be very useful probes for studying the physiological functions of these enzymes. In better understanding these physiological functions, more effective therapies may be developed. To date, a large number of PTKs and PTPs have been identified but their precise mechanisms of action in vivo have been largely unknown.

A variety of PTP inhibitors have been reported in recent years (reviewed in 5). Virtually all of these inhibitors contain a nonhydrolyzable pY mimetic, such as phosphonates (6-10), malonates (11-14), aryl carboxylates (15-19), or cinnamates (20, 21), as the inhibitor core structure. Although some of these inhibitors have shown impressive potency in vitro (10, 19, 20), their application as therapeutic agents or research tools in whole cell assays has yet to be realized. A few natural (22, 23) and synthetic products (24) have also been found to show inhibitory activity toward PTPs, but they are generally weak inhibitors.

As recent research has shown, malfunctioning PTPs and PTKs are responsible for causing a variety of human conditions and diseases, such as type II diabetes. There exists a need for new pY mimetics which are able to inhibit particular PTPs, such as PTP-1B, which has been linked to type II diabetes. Furthermore, a large number of PTPs and PTKs that have been identified, but their in vivo mechanisms of action have not been studied due to the lack of suitable pY mimetics. There exists a need for new, pY mimetics that can be used to study the functions and mechanisms of individual PTPs and PTKs.

### SUMMARY

Methods of reversibly inhibiting protein tyrosine phosphatases (PTPs) and Src homology 2 (SH2) domains are provided. The methods comprise contacting the PTP or SH2 domains with a neutral phosotyrosine (pY) mimetic (the PTP inhibitor) comprising an aldehyde or mono-ketone substituted aryl group. The aldehyde or mono-ketone of the pY mimetics form a reversible imine or enamine adduct with the active site arginine of the PTP. The aryl group provides the pY mimetic with molecular affinity to the active site of the PTP. Finally, a second substituent on the aryl group is tailored to interact with the reactive surface near the active site of the PTP through a host of molecular interactions, including any of: electrostatic interactions, hydrogen bonding, hydrophobic interactions, and van der Waals interactions. Because of the reversible interaction between the active site arginine and the first reactive group of the pY mimetic to form an imine or enamine adduct, the present methods may be tailored to any PTP currently known or later discovered. Since the area around the active site is different in different PTPs, by changing the second reactive group, which interacts with that area, inhibitors with specificity to individual PTPs, such as PTP-1B, SHP-1, VHR, and CD45, can be made. Further improvements may be made to the inhibitors through further derivatization of the second reactive group.

A general structure of a pY mimetic is shown in Formula I:

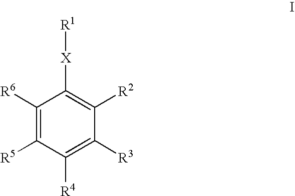

wherein X is selected from the group consisting of C, N, O, and S; provided that when X is N or C, X may be further substituted with a radical selected from the group consisting of H, halo, C₁-C₇ alkyl, aryl, arylalkyl, and C₁-C₇ alkoxy. R¹ is selected from the group consisting of C₁-C₇ aldehyde and C₁-C₇ ketone; and R₁ is optionally substituted at any substitutable position with H, halo, aryl, arylalkyl, C₁-C₇ alkyl, C₁-C₇ haloalkyl, or C₁-C₇ alkoxy. R²-R⁶ are selected from the group consisting of H, F, Cl, Br, I, NO₂, CN, OH, C₁-C₂₀ alkyl, C₁-C₂₀ alkenyl, aryl, alkylaryl, arylalkyl, C₁-C₂₀ alkoxy; R⁷OCO—, R⁷COO—, R⁷NHCO—, R⁷CONH—, peptidyl arylpeptidyl, and combinations thereof; wherein R⁷ is selected from H, C₁-C₂₀ alkyl, C₁-C₂₀ alkenyl, aryl, alkylaryl, arylalkyl, and C₁-C₂₀ carboxylic acids. Optionally, two of R²-R⁶ on adjacent C atoms may be joined to form a ring structure, wherein a fused polycyclic structure will be formed, the ring structure selected from the group consisting of aromatic; heterocyclic aromatic; saturated carbocyclic, saturated heterocyclic, partially saturated carbocyclic, and partially saturated heterocyclic.

A preferred class of compounds within Formula I is the class wherein the base molecule is cinnamaldehyde, as depicted in Formula II:

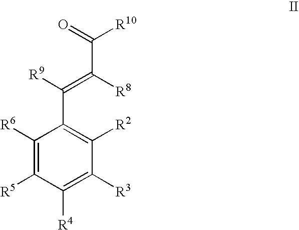

wherein R²-R⁶ are selected from the group consisting of H, F, Cl, Br, I, NO₂, CN, OH, C₁-C₂₀ alkyl, C₁-C₂₀ alkenyl, aryl, alkylaryl, arylalkyl, C₁-C₂₀ alkoxy; R⁷OCO—, R⁷COO—, R⁷NHCO—, R⁷CONH—, peptidyl, arylpeptidyl and combinations thereof; wherein R⁷ is selected from H, C₁-C₂₀ alkyl, C₁-C₂₀ alkenyl, aryl, alkylaryl, arylalkyl, and C₁-C₂₀ carboxylic acids. R⁸ and R⁹ are selected from the group consisting of H, halo, C₁-C₇ alkyl, and C₁-C₇ alkoxy and R¹⁰ is selected from the group consisting of H, C₁-C₇ alkyl, benzyl, C₁-C₇ haloalkyl, and C₁-C₇ alkoxymethyl.

Another preferred class of compounds useful for inhibiting PTPs are compounds in which the aryl component has a fused ring which could be another aromatic ring, thus forming a fused aromatic, or another saturated or partially saturated ring. These compounds are shown in Formula III:

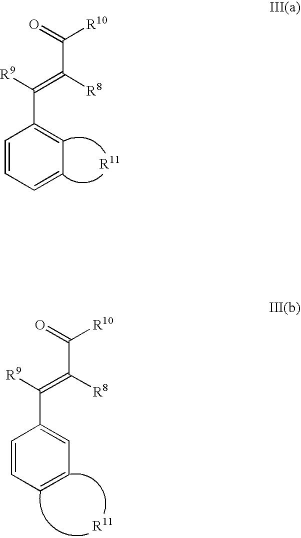

wherein R⁸ and R⁹ are selected from the group consisting of H, halo, C₁-C₇ alkyl, and C₁-C₇ alkoxy and R¹⁰ is selected from the group consisting of H, C₁-C₇ alkyl, benzyl, C₁-C₇ haloalkyl, and C₁-C₇ alkoxymethyl. R¹¹ is a fused ring selected from the group consisting of aromatic; heterocyclic aromatic; saturated carbocyclic, saturated heterocyclic, partially saturated carbocyclic, and partially saturated heterocyclic. The fused rings of Formula III can have further substituents on the rings to provide further affinity for the active site of the particular PTP of interest.

Another preferred class of compounds is depicted in Formula IV:

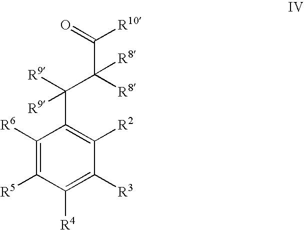

wherein R²-R⁶ are selected from the group consisting of H, F, Cl, Br, I, NO₂, CN, OH, C₁-C₂₀ alkyl, C₁-C₂₀ alkenyl, aryl, alkylaryl, arylalkyl, C₁-C₂₀ alkoxy; R⁷OCO—, R⁷COO—, R⁷NHCO—, R⁷CONH—, peptidyl arylpeptidyl, and combinations thereof; wherein R⁷ is selected from H, C₁-C₂₀ alkyl, C₁-C₂₀ alkenyl, aryl, alkylaryl, arylalkyl, and C₁-C₂₀ carboxylic acids. R^{8′} and R^{9′} are selected from the group consisting of H, halo, C₁-C₇ alkyl, and C₁-C₇ alkoxy and combinations thereof. R^{10′} is selected from the group consisting of H, C₁-C₇ alkyl, benzyl, C₁-C₇ haloalkyl, and C₁-C₇ alkoxymethyl.

A fourth preferred class of compounds within Formula I are those of Formula V:

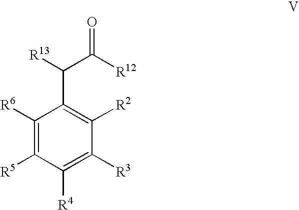

wherein R²-R⁶ are selected from the group consisting of H, F, Cl, Br, I, NO₂, CN, OH, C₁-C₂₀ alkyl, C₁-C₂₀ alkenyl, aryl, alkylaryl, arylalkyl, C₁-C₂₀ alkoxy, R⁷OCO—, R⁷COO—, R⁷NHCO—, R⁷CONH—, peptidyl, arylpeptidyl, and combinations thereof; wherein R⁷ is selected from H, C₁-C₂₀ alkyl, C₁-C₂₀ alkenyl, aryl, alkylaryl, arylalkyl, and C₁-C₂₀ carboxylic acids. R¹² is selected from the group consisting of H, C₁-C₇ alkyl, benzyl, C₁-C₇ haloalkyl, and C₁-C₇ alkoxymethyl; and R¹³ is selected from the group consisting of H, halo, C₁-C₇ alkyl, and C₁-C₇ alkoxy.

A fifth preferred class of compounds within Formula I are those compounds in which the aldehyde or mono-ketone are connected through a heteroatom. These compounds are shown in Formula VI:

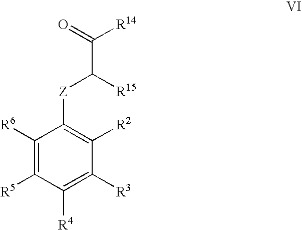

wherein R²-R⁶ are selected from the group consisting of H, F, Cl, Br, I, NO₂, CN, OH, C₁-C₂₀ alkyl, C₁-C₂₀ alkenyl, aryl, alkylaryl, arylalkyl, C₁-C₂₀ alkoxy, R⁷OCO—, R⁷COO—, R⁷NHCO—, R⁷CONH—, peptidyl, arylpeptidyl and combinations thereof; wherein R⁷ is selected from H, C₁-C₂₀ alkyl, C₁-C₂₀ alkenyl, aryl, alkylaryl, arylalkyl, and C₁-C₂₀ carboxylic acids. R¹⁴ is selected from the group consisting of consisting of H, C₁-C₇ alkyl, benzyl, C₁-C₇ haloalkyl and C₁-C₇ alkoxymethyl; and R¹⁵ is selected from the group consisting of H, halo, C₁-C₇ alkyl, and C₁-C₇ alkoxy. Z is selected from the group consisting of O, S, or NH.

Also included in the compounds of Formula I are compounds in which the second reactive group is a tripeptide, as in Formulae VII, VIII, and IX:

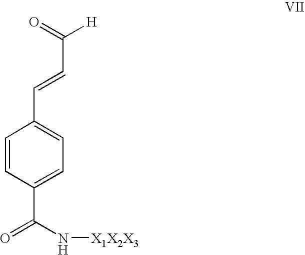

wherein X₁X₂X₃ is a tripeptide formed from α-amino acids. When the pY mimetic is used to inhibit PTPIB, especially preferred tripeptides are: PVL, NSV, QLL, FPS, NIY, AAF, NLG, APL, PQH, PQL, MLF, EVM, YYT, QMP, APP, NAS, GFQ, ILE, TPH, FEA, YIF, VRR, LRF, DVK, NPH, RKR, VRK, GLW, FRL, RFR, LRL, GNR, FRG, GRL, LTR, YRK, SYR RRL, GRR, SRF, LYR, YNR, RTR, TLR, GYY, LRF, NFW, TRL, YRL, RFL, FRR, RGR, RFY, NRF, GLR, DRR, NRF, RLR, RVF, ARR, FRL, DRR, TRR, RVR, RRK, FRL, QLR, RNV, NPW, FRL, FPR, GRF, and SKR. When the pY mimetic is used to inhibit the SHP-1 catalytic domain, preferred tripeptides are: YWY, INE, VSH, LPL, VLY, VDH, DHG, LLF, LDE, EDM, VLE, DTA, VSN, QGE, SME, FVQ, PAL, QDS, NTL, EAY, FML, IIH, VYN, NFI, VPG, GDV, and HQE. When the pY mimetic is used to inhibit the N-SH2 domain of SH-1, preferred tripeptides are: VHL, YTR, DRN, RLQ, EEY, NDS, RGR, and RML.

The second tripeptide-substituted inhibitor is represented by Formula VIII:

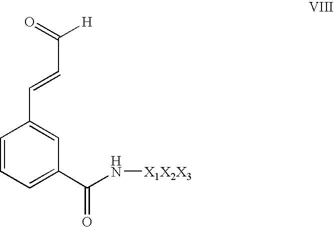

wherein the tripeptide chosen for pY mimetics used to inhibit PHPIB is selected from QTQ, EGP, IHV, YNH, QVT, GVN, PVY, and PFL. The tripeptide chosen for pY mimetics to inhibit SHP-1 is selected from DGL, AYV, EVA, VDL, TYG, SII, LED, QAL, QYP, VTI, and MMM. The tripeptide chosen for pY mimetics used for the SHP-1 SH-2 domain is selected from APL, RWL, ARN, GRT, RRV, VAR, PLL, IAH, NSR, IKL, LRR, DVR, IEF, EYR, IRF, and VKR.

The third tripeptide-substituted pY mimetic of the present invention is represented by Formula IX:

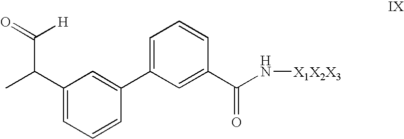

When the pY mimetic of Formula IX is used to inhibit PHP1B, preferred tripeptides are selected from YRY, VDW, RWR, VWA, VAR, DKA, GGA, DFL, LYM, YPY, YRL, VRM, VRF, LKW, IRF, RSF, WFL, RGR, EGA, EFP, YYR, WKV, VAW, WLR, VLL, YYR, NHY, SFW, YPL, RRA, YSP, FVG, ALG, SWA, GGA, GFN, FEY, ENV, MLM, NVS, VYM, YSL, AEN, EHL, LVY, VEM, VYT, GPT, and GTE. When the pY mimetic of Formula IX is used to inhibit SHP-1 preferred tripeptides are selected from SYF, VLF, VLV, QPF, YPA, AVA, IGP, HHA, SYP, FGA, IVT, QVS, QLV, TFH, GQY, YMI, and VVS. When the pY mimetic of Formula IX is used to inhibit the N-SH2 domain of SHP-1, preferred tripeptides are selected from the group consisting of EDY, RTH, EHV, NYP, VVT, HIH, EVF, KQI, ILR, PYY, HRM, SQY, KVR, LHF, and VHV.

Method of inhibiting phosphotyrosine proteases (PTPs) and Src homology 2 (SH2) domains by targeting the active site arginine in the PTPs and SH2 domains are provided. Neutral, reversible PTP and SH2 domain inhibitors that can be derivatized to enhance the molecular interactions between the inhibitor, or pY mimetic and the active site, and the inhibitor and the surface surrounding the active site are also provided. Methods of using the neutral, reversible inhibitors as probes for studying the physiological functions of PTPs and SH2 domains are also provided. Methods of treating type II diabetes and obesity by administering a neutral pY mimetic that selectively and reversibly binds with PTP1B are also provided. Neutral, reversible inhibitors of SH2 domains, having applications in treating cancer, osteoporosis, and inflammation are also provided.

### BRIEF DESCRIPTION OF THE FIGURES

FIG. 1. Structures of PTP inhibitors.

FIG. 2. Plot of remaining PTP activity against inhibitor 3 concentration. All of the activities are relative to those in the absence of inhibitor.

FIG. 3. Slow-binding inhibition of PTP1B by Cinn-GEE. (A) Hydrolysis of pNPP (1.0 mM) by PTP1B (0.2 μM) in the presence of indicated amounts of Cinn-GEE. The reactions were initiated by the addition of enzyme as the last component. (B) Hydrolysis of pNPP by reactivated PTP1B. The enzyme (1.0 μM) was preincubated with Cinn-GEE (75 μM) for 3 h before being diluted 10-fold into the reaction buffer containing 1.0 mM pNPP.

FIG. 4. HSQC spectra of ¹³C-labeled Cinn-GEE in the presence and absence of PTP1B. (A) 200 μM Cinn-GEE only; (B) 200 μM Cinn-GEE and ˜200 μM PTP1B; and (C) 200 μM Cinn-GEE and ˜300 μM PTP1B. PTP1B concentration was based on the Bradford assay using bovine serum albumin as standard.

FIG. 5. Inactivation of Cinn-GEE by cysteamine. PTP1B (0.2 μM) was preincubated with Cinn-GEE alone (20 μM), cysteamine alone (200 μM), or Cinn-GEE (20 μM) and cysteamine (200 μM) before being added to the reaction mixture containing 1.0 mM pNPP (pH 7.4).

FIG. 6. Proposed mechanism of inhibition of PTP by cinnamaldehyde derivatives. X, a PTP side chain or a water molecule. The asterisk indicates the position of ¹³C labeling.

### DETAILED DESCRIPTION

The methods of inhibiting PTPs of the present invention have applications ranging from treatments for type II diabetes and obesity as a function of inhibiting PTP-1B, to treatments for cancer, osteoporosis, and inflammation as a function of inhibiting the Src homology 2 (SH2) domains of specific proteins, through the utilization of neutral phosphotyrosine (pY) mimetics. Because the pY mimetics of the present invention are neutral, the inhibitors have good membrane permeability, unlike other pY mimetics that have been studied.

There are at least 56 PTPs in human, identified through genomic sequencing. PTPs were identified and studied relatively recently, with the first one being purified in 1988 (PTP1B). Therefore, the physiological and pathological functions of PTPs are for the most part unknown.

A few PTPs have been better studied including PTP1B, SHP-1, and CD45. PTP1B has been demonstrated as a viable target for treating type II diabetes and obesity (4). CD45 is expressed exclusively by hematopoietic cells and is required for T cell activation. Inhibitors of CD45 are expected to provide potential treatment for transplant rejection and autoimmune diseases (Urbanek et al. J. Med. Chem. 2001, 44, 1777). SHP-1 is also expressed primarily by hematopoietic cells. It comprises two tandem SH2 domains at the N-terminus and a PTP domain at the C-terminus. It is involved in many signaling pathways. Mutation of SHP-1 in mice results in the deregulation of their immune system, with symptoms of both immunodeficiency and autoimmune disease.

Many more PTPs are expected to be found in disease processes as the functions of additional PTPs are elucidated. The pY mimetics used in the methods of the present invention are expected to be useful against all of the PTPs, which have a very conserved active site.

The methods of the present invention also. provide effective research tools for studying the large number of PTKs and PTPs that have been found, but heretofore have been difficult to study because of the lack of neutral, reversible pY mimetics.

One of the biggest challenges right now in the signal transduction field is to find out the targets of PTPs. Selective PTP inhibitors would allow us to specifically inhibit a single PTP in a cell and examine the consequences, from which one can gleam the functions of the particular PTP. While a number of non-selective PTP inhibitors are known, selective inhibitors are needed to better understand the functions of particular PTPs.

The pY mimetics of the present invention are also useful as inhibitors against SH2 domains. There are at least 95 known SH2 domains in humans. Their function is to bind to a pY peptide in a partner protein, thereby bringing the two proteins together. They do so by binding to pY plus three residues immediately C-terminal to pY. A typical SH2 domain has two main binding pockets, one for binding pY and another for binding to one of the three C-terminal residues. The other two C-terminal residues can also make surface contacts with SH2.

Both genetic studies and in vivo studies with SH2 inhibitors have shown that inhibition of the SH2 domain of Src kinase can inhibit bone resorption by osteoclasts. Therefore, Src SH2 inhibitors are expected to provide treatment for bone thinning (osteoporosis). In addition, inhibitors may also be useful for treating a number of cancers. Likewise, SH2 inhibitors against Grb2 SH2 domain are expected to be useful for treating cancers (reviewed by Shakespeare, Current Opinion in Chemical Biology 2001, 5, 409).

Like PTP inhibitors, SH2 domain inhibitors will be useful in identifying the physiological targets of these SH2-containing proteins.

The catalytic SHP-1 is autoinhibited by its own N-SH2 domain, which binds directly to and inactivates the PTP domain. When a ligand, such as pY or an antagonist binds to the N-SH2 domain, the intramolecular complex is disrupted, thereby relieving the catalytic domain from auto-inhibition. Therefore, a mimetic that can bind to the N-SH2 domain will be able to stimulate the activity of the SHP-1.

The terms “therapeutically effective” and “pharmacologically effective” are intended to qualify the amount of each agent which will achieve the goal of improvement in disease severity and the frequency of incidence over treatment of each agent by itself, while avoiding adverse side effects typically associated with alternative therapies.

The term “subject” for purposes of treatment includes any human or animal subject who has a disorder caused by the malfunction of PTPs or SH2 domains, or would benefit from treatment with a selective PTP inhibitor. Such disorders include, but are not limited to type II diabetes, obesity, immunodeficiency, autoimmune disease, osteoporosis, and cancers. Subjects also include transplant recipients, as the PTP inhibitors can be developed into therapeutics which will help prevent transplant rejection. For methods of prevention the subject is any human or animal subject, and preferably is a human subject who is at risk of developing one of the above- mentioned disorders, or a person who is receiving a transplant. Besides being useful for human treatment, the compounds of the present invention are also useful for veterinary treatment of mammals, including companion animals and farm animals, such as, but not limited to dogs, cats, horses, cows, sheep, and pigs. Preferably, subject means a human.

In accordance with the methods of the present invention, formation of reversible adduct with active-site arginine to form imine or enamine, including, but not limited to, aldehydes and monoketones. It does not include 1,2-diketones or acyl halides. The methods of the present invention use PTP inhibitors that alkylate the active-site arginine (Arg221 in PTP1B or its equivalent in other PTPs). The inhibitors generally do not react with any other arginine, or with active-site cysteine.

Derivatives are intended to encompass any compounds which are structurally related to the compounds of formulae I-IX or which possess the substantially equivalent activity, as measured by the derivative's ability to form a reversible imine or enamine adduct with the active site arginine in PTPs and SH2 domains. By way of example, such compounds may include, but are not limited to, prodrugs thereof. Such compounds can be formed in vivo, such as by metabolic mechanisms.

The present invention also relates to therapeutic methods of treating disorders caused by PTP mutations, such as type II diabetes and obesity. The methods comprise administering a therapeutically effective amount of a compound of formula I or II to a subject having a disorder or being predisposed to a disorder caused by a mutation to a PTP.

As used herein, the terms “pY mimetic” and “PTP inhibitor” are interchangeable. The compounds of the present invention are pY mimetics, and as such, function to inhibit PTPs through the reversible formation of an adduct with the active site arginine. The same compounds also form a reversible adduct with SH2 domains, which also have a conserved arginine in the active site. Since the compounds used in the methods of the present invention perform both functions, the two terms are used interchangeably.

Where the term alkyl is used, either alone or with other terms, such as haloalkyl or alkylaryl, it includes C₁ to C₂₀ linear or branched alkyl radicals, examples include methyl, ethyl, propyl, isopropyl, butyl, tert-butyl, and so forth. The term “haloalkyl” includes C₁ to C₁₀ linear or branched alkyl radicals substituted with one or more halo radicals. Some examples of haloalkyl radicals include trifluoromethyl, 1,2-dichloroethyl, 3-bromopropyl, and so forth. The term “halo” includes radicals selected from F, Cl, Br, and I.

The term aryl, used alone or in combination with other terms such as alkylaryl, haloaryl, or haloalkylaryl, includes such aromatic radicals as phenyl, biphenyl, and benzyl, as well as fused aryl radicals such as naphthyl, anthryl, phenanthrenyl, fluorenyl, and indenyl and so forth. The term “aryl” also encompasses “heteroaryls,” which are aryls that have carbon and one or more heteroatoms, such as O, N, or S in the aromatic ring. Examples of heteroaryls include indolyl, pyrrolyl, and so on. “Alkylaryl” or “arylalkyl” refers to alkyl-substituted aryl groups such as butylphenyl, propylphenyl, ethylphenyl, methylphenyl, 3,5-dimethylphenyl, tert-butylphenyl and so forth. “Haloaryl” refers to aryl radicals in which one or more substitutable positions has been substituted with a halo radical, examples include fluorophenyl, 4-chlorophenyl, 2,5-chlorophenyl and so forth. “Haloalkylaryl” refers to aryl radicals that have a haloalkyl substituent. Examples of haloalkylaryls include such radicals as bromomethylphenyl, 4-bromobutylphenyl and so on.

Also included in the family of compounds of formulae I-IX are the pharmaceutically acceptable salts thereof. The phrase “pharmaceutically acceptable salts” connotes salts commonly used to form alkali metal salts and to form addition salts of free acids or free bases. The nature of the salt is not critical, provided that it is pharmaceutically acceptable. Suitable pharmaceutically acceptable acid addition salts of compounds of formulae I-IX may be prepared from an inorganic acid or from an organic acid. Examples of such inorganic acids are hydrochloric, hydrobromic, hydroiodic, nitric, carbonic, sulfuric, and phosphoric acid. Appropriate organic acids may be selected from aliphatic, cycloaliphatic, aromatic, araliphatic, heterocyclic, carboxylic, and sulfonic classes of organic acids, examples of which include formic, acetic, propionic, succinic, glycolic, gluconic, lactic, malic, tartaric, citric, ascorbic, glucoronic, maleic, fumaric, pyruvic, aspartic, glutamic, benzoic, anthranilic, mesylic, salicylic, p-hydroxybenzoic, phenylacetic, mandelic, ambonic, pamoic, methanesulfonic, ethanesulfonic, benzenesulfonic, pantothenic, 2-hydroxyethanesulfonic, toluenesulfonic, sulfanilic, cyclohexylaminosulfonic, stearic, algenic, β-hydroxybutyric, galactaric, and galacturonic acids. Suitable pharmaceutically acceptable base addition salts of compounds of formulae I-IX include metallic salts made from aluminum, calcium, lithium, magnesium, potassium, sodium, and zinc. Alternatively, organic salts made from N,N′-dibenzylethylenediamine, chloroprocaine, choline, diethanolamine, ethylenediamine, meglumine (N-methylglucamine) and procaine may be used form base addition salts of the compounds of formulae I-IX. All of these salts may be prepared by conventional means from the corresponding compounds of formulae I-IX by reacting, for example, the appropriate acid or base with the inhibitor of the present invention.

All of the known PTPs contain an invariant sequence motif at the active site, (I/V)HC(X)₅R(S/T), where the cysteine and arginine residues are strictly conserved. Mechanistically, the cysteine thiol is the catalytic nucleophile, which attacks the phosphate of pY to form a transient phosphocysteinyl enzyme intermediate (25). The arginine is critical for both substrate binding and stabilization of the transition state by neutralizing the developing negative charge on the phosphate group (26). Mutation or chemical modification of the active-site cysteine results in total loss of activity (27, 28), whereas removal of the arginine side chain reduces the activity by >10⁴-fold (26). It was previously demonstrated that α-haloacetophenone derivatives act as potent, time-dependent inactivators of PTPs by alkylating the active-site cysteine (29). It has now been discovered that simple aldehydes act as slow-binding, reversible inhibitors of PTPs presumably by forming an imine or enamine with the active-site arginine.

All examples disclosed herein are for illustrative purposes only and are not meant to limit the claimed invention in any way.

### EXAMPLES

### Example 1

### Materials and General Methods

The catalytic domain of SHP-1, SHP-1(ΔSH2), was purified from a recombinant Escherichia coli strain as previously described (30). PTP1B and VHR were overexpressed in E. coli and purified according to literature procedures (31, 32). All of the peptide synthesis reagents were purchased from Advanced ChemTech (Louisville, Ky.). The Rink resin had a loading capacity of 0.7 mmol/g. All other chemicals were obtained from Aldrich or Sigma.

Methyl 4-[2-(1,3-Dioxolane-2-yl)-ethenyl]benzoate (6): This compound was prepared by modification of a literature procedure (33). Methyl 4-formylbenzoate (0.82 g, 5.0 mmol) and tris(methoxyethoxyethyl)amine (TMA) (1.62 g, 5.0 mmol) were dissolved in 30 mL of dichloromethane under argon at room temperature. A saturated aqueous K₂CO₃ solution (30 mL) and (1,3-dioxolane-2-yl-methyl)triphenylphosphonium bromide (2.15 g, 5 mmol) were added, and the reaction mixture was refluxed (˜40° C.) for 16 h with vigorous stirring. The reaction product was extracted into dichloromethane (3×20 mL), washed with water (20 mL), and dried over MgSO₄. Compound 6 was obtained as a 67:33 mixture of Z and E isomers after chromatography on a silica gel column (6:1 hexane/ethyl acetate) (yield: 1.16 g, 99.1%). ¹H NMR (250 MHz, CDCl₃) δ 7.97-8.10 (m, 2H, Ar), 7.52-7.76 (m, 2H, Ar), 6.83-6.93 (m, 1H, 0.33H, ArCH═CHCH E, J=5.9 Hz), 3.88-4.18 (m, 7H, CH₃, OCH₂CH₂O); ¹³C NMR (62.5 MHz, CDCl₃) δ 166.63, 141.31, 134.39, 133.47, 131.39, 130.58, 130.13, 129.83, 127.64, 104.06, 99.98, 65.76, 65.61, 52.31.

Methyl 4-(3-Oxo-1-propenyl)benzoate (9). Compound 6 (0.12 g, 0.5 mmol) was added to 5 mL of 90% aqueous TFA and the solution was stirred for 1 h. After removal of solvents by rotary evaporation, the residue was dried over P₂O₅ in vacuo to a white solid as pure E isomer (95 mg, quantitative yield). ¹H NMR (250 MHz, CDCl₃) δ 9.74 (d, 1H, CHO, J=7.5 Hz), 8.09 (d, 2H, Ar, J=8.4 Hz), 7.87 (d, 2H, Ar, J=8.4 Hz), 7.77 (d, 1H, ArCH═CHCH, J=16.1 Hz), 6.87 (dd, 1H, ArCH═CHCH, J=7.5 Hz, 13.3 Hz), 3.91 (s, 3H, OCH₃); ¹³C NMR (62.5 MHz, CDCl₃) δ 194.12, 166.61, 151.62, 132.86, 131.44, 129.47, 52.59.

4-(3-Oxo-1-propenyl)benzoic acid (3). Compound 9 (48 mg) was added to 3 mL of an aqueous NaOH solution (40 mg, 1 mmol) and the solution was stirred overnight at room temperature. After removal of methanol by rotary evaporation, the remaining solution was diluted with 5 mL of H₂O and acidified to pH 2 with HCl. The solution was extracted with ethyl acetate (3×10 mL) and the combined organic phase was dried over MgSO₄. Evaporation of solvent afforded 41 mg of a light yellow solid (92.4% yield). ¹H NMR (250 MHz, CDCl₃) δ 10.46 (s, 1H, COOH), 9.78 (d, 1H, CHO, J=7.6 Hz), 8.14 (d, 2H, Ar, J=8.3 Hz), 7.88 (d, 2H, Ar, J=8.3 Hz,), 7.78 (d, 1H, ArCH═CHCH, J=16.1 Hz), 6.89 (dd, 1H, ArCH═CHCH, J=7.5 Hz, 16.0 Hz); ¹³C NMR (62.5 MHz, CDCl₃) δ 194.15, 164.15, 151.24, 134.52, 131.07, 129.45.

Ethyl 4-[2-(1,3-dioxolane-2-yl)-ethenyl]benzoyl carbonate (7). An aqueous NaOH solution (0.8 g dissolved in 8 mL of H₂O) was added to compound 6 (1.16 g, 5.0 mmol) dissolved in 8 mL of methanol. The solution was stirred for 16 h at room temperature, followed by solvent evaporation. The solid residue was dried over P₂O₅ in vacuo to produce the corresponding sodium salt, which was stable during storage in a −5° C. freezer. Ethyl chloroformate (0.26 g, 24 mmol) was added to a 15-mL suspension of the salt (0.14 g) in CH₂Cl₂ under argon and the mixture was stirred for 2 h at room temperature and filtered. The filtrate was evaporated under reduced pressure to obtain 0.12 g of an oil (isomer Z:E=62:38) (86% yield). ¹H NMR (250 MHz, CDCl₃) δ 8.02-8.08 (m, 2H, Ar), 7.60-7.72 (m, 2H, Ar), 6.86-6.92 (m, 1H, ArCH═CHCH), 6.42 (dd, 0.38H, ArCH═CHCH E, J=5.8 Hz, 16.1 Hz), 5.86 (dd, 0.62H, ArCH═CHCH Z, J=7.4 Hz, 11.8 Hz), 5.46 (d, 0.62H, ArCH═CHCH Z, J=7.4 Hz), 5.41 (d, 0.38H, ArCH═CHCH E, J=5.9 Hz), 4.29-4.42 (m, 2H. CH₂CH₃), 3.91-3.94 (m, 4H, OCH₂CH₂O), 1.29-1.38 (m, 3H, CH₂CH₃); ¹³C NMR (62.5 MHz, CDCl₃) δ 161.80, 149.93, 149.30, 143.33, 143.09, 134.00, 132.24, 131.57, 131.28, 131.11, 130.99, 130.39, 128.19, 127.85, 127.57, 103.85, 99.85, 66.71, 65.79, 65.63, 14.15.

N-[4-(3-Oxo-1-propenyl)benzoyl]-Gly-Glu-Glu-NH₂ (5). The tripeptide Gly-Glu-Glu was synthesized on 0.18 g of Rink resin (0.11 mmol) using standard Fmoc/HBTU/HOBt solid-phase peptide chemistry. Next, 61.3 mg (0.21 mmol) of anhydride 7 in 2.5 mL CH₂Cl₂ was added to the resin suspended in 2.5 mL of anhydrous DMF. The mixture was shaken for 8 h at room temperature. Ninhydrin tests indicated complete acylation of the N-terminal amine. The solvents were drained and the resin was washed with DMF (5×5 mL) and CH₃OH (3×5 mL). Deprotection of side-chain as well as aldehyde protecting groups and cleavage from the resin were carried out with a cocktail containing 4 mL of 90% TFA in water, 0.1 mL of anisole, and 0.15 mL of thioanisole for 3 h at room temperature. TFA, H₂O and other volatile chemicals were removed under a gentle flow of nitrogen, and the residue was triturated five times with diethyl ether. Compound 5 was obtained as a brownish solid in the pure E isomer form (yield 48 mg). ¹H NMR (250 MHz, D₂O): 9.62 (d, CHO, J=7.4 Hz), 7.79-7.86 (m, Ar, ArCH═CHCH), 6.87 (dd, ArCH═CHCH, J=7.5 Hz, 16.0 Hz), 4.11-4.42 (m, α-CH of Glu), 3.84 (s, α-CH₂ of Gly), 2.37-2.42 (m, 2 CH₂CH₂COOH), 1.96-2.12 (m, 2 CH₂CH₂COOH). HRESI-MS: C₂₂H₂₆O₉N₄Na⁺ ([M+Na]⁺), calcd. 513.1592, found 513.1576.

Methyl 4-(3-[¹³C]Oxo-1-propenyl)benzoate (10). (Triphenylphosphoranylidene)-[1-¹³C]acetaldehyde (11) was synthesized as previously described (34). The crude aldehyde 11 (0.85 g, 2.8 mmol) after crystallization was dissolved in 25 mL of benzene and mixed with methyl 4-formyl benzoate (0.7 g, 4.3 mmol). The solution was stirred for 6 h at room temperature and the solvent was removed by rotary evaporation. Chromatography on a silica gel column (hexane/ethyl acetate=6:1) afforded 0.17 g of a white solid (26% yield for two steps). ¹H NMR (250 MHz, CDCl₃): δ 9.75 (dd, 1H, ¹³CHO, ³J_{H-H}=7.6 Hz, ¹J_{C-H}=173.1 Hz), 8.09 (d, 2H, Ar, ³J_{H-H}=6.7 Hz), 7.64 (d, 2H, Ar, ³J_{H-H}=8.4 Hz), 7.50 (d, 1H, ArCH═CHCH, ³J_{H-H}=16.0 Hz, ³J_{C-H}33.5 Hz), 6.78 (ddd, 1H, ArCH═CHCH E, ³J_{H-H}=7.5 Hz, 16.0 Hz, ²J_{C-H}=0.85 Hz), 3.95 (s, 3H, OCH₃); ¹³C NMR (62.5 MHz, CDCl₃) δ 6 193.65.

Methyl 4-[2-(1,3-Dioxolane-2-[¹³C]yl)-ethenyl]benzoate (12). A solution of ethylene glycol (0.11 g, 1.78 mmol) in benzene (5 mL) was added into a round-bottomed flask containing compound 10 (0.17 g, 0.89 mmol), p-toluenesulfonic acid monohydrate (3 mg), MgSO₄ (0.22 g, 1.78 mmol), and benzene (25 mL). The reaction mixture was heated to reflux for 8 h. After cooling, solid NaHCO₃ (5.4 mg, 0.064 mmol) was added to the mixture to neutralize the p-toluenesulfonic acid and the mixture was stirred for 30 min. The reaction mixture was then filtered through a pad of anhydrous NaHCO₃ and the filter cake was washed with CH₂Cl₂ (3×10 mL). The filtrate was concentrated and purified by flash column chromatography on a silica gel column (6:1 hexane/ethyl acetate) to obtain 0.14 g of a white solid (67% yield). ¹H NMR (250 MHz, CDCl₃) δ 7.99 (d, 2H, Ar, ³J_{H-H}=6.6 Hz), 7.63 (d, 2H, Ar, ³J_{H-H}=6.6 Hz,), 6.87 (dd, 1H, ArCH═CHCH, ³J_{H-H}=16.0 Hz, ³J_{C-H}=6.7 Hz), 6.37 (dd, 1H, ArCH═CHCH E, ³J_{H-H}=5.8 Hz, 16.0 Hz), 5.39 (dd, 1H, ³J_{H-H}=5.9 Hz, 2J_{C-H}=167.43 Hz), 3.88-4.04 (m, 7H, OCH₃, OCH₂CH₂O); ¹³C NMR (62.5 MHz, CDCl₃) δ 104.08.

Ethyl 4-[2-(1,3-dioxolane-2-[¹³C]yl)-ethenyl]benzoyl carbonate (13). This was prepared as described for 7. ¹H NMR (250 MHz, CDCl₃) δ8.03 (d, 2H, Ar, ³J_{H-H}=8.0 Hz), 7.51 (d, 2H, Ar, ³J_{H-H}=8.05 Hz), 6.82 (dd, 1H, ArCH═CHCH, ³J_{H-H}=15.8 Hz, ³J_{C-H}=7.45 Hz), 6.31 (dd, 1H, ArCH═CHCH E, ³J_{H-H}=5.6 Hz, 15.9 Hz), 5.46 (dd, IH, ³J_{H-H}=5.6 Hz, ²J_{C-H}=168.1 Hz), 4.40 (m, 2H, OCH₂CH₃), 3.96-4.09 (m, 4H, OCH₂CH₂O), 1.44 (t, 3H, OCH₂CH₃); ¹³C NMR (62.5 MHz, CDCl₃) δ 103.52.

N-[4-(3-[¹³C]Oxo-1-propenyl)benzoyl]-Gly-Glu-Glu-NH₂ (14). This was prepared from anhydride 13 in a similar manner to 5. ¹³C NMR (62.5 MHz, CDCl₃) δ 194.43.

PTP Inhibition Assays. Stock solutions of inhibitors were prepared in dimethyl sulfoxide (DMSO) and their concentrations were calculated from the known inhibitor masses and solvent volumes. A typical reaction (total volume 1 mL) contained 50 mM HEPES (pH 7.4), 1 mM EDTA, 1 mM tris(carboxyethyl)phosphine (TCEP), 50 mM NaCl, 5% (v/v) DMSO, 0.1-0.2 μM PTP, and 0-2000 μM inhibitor. After incubation of the enzyme with the inhibitor for 1 h at room temperature, the reaction was initiated by the addition of 1.0 mM p-nitrophenyl phosphate (pNPP). The reaction progress was monitored at 405 nm on a Perkin-Elmer UV-Vis spectrophotometer. The IC₅₀ values were determined by plotting the remaining activity as a function of inhibitor concentration, and the K₁* values were obtained by fitting the data to the Michaelis-Menten equation. To determine the K₁ value, the reaction was initiated by addition of enzyme (0.2 μM) into the above reaction mixture, which also contained 1.0 mM pNPP. The reaction was monitored continuously on the UV-Vis spectrophotometer and the initial reaction rates (<15 s) were fitted to the Michaelis-Menten equation.

To determine the rate constant κ₆, the enzyme (1.0 μM) was incubated with 50-100 μM inhibitor in 100 μL of the above reaction buffer for 3 h at room temperature. The mixture was rapidly diluted into 900 μL of the same reaction buffer containing 1.0 mM pNPP (without inhibitor). Reactivation of PTP activity was monitored at 405 nm and the progress curves were fit to the equation

Abs₄₀₅=νₛ[t−(1−e^{-κ6t})/κ₆]

where νₛ is the final steady-state velocity. The rate constant κ₅ was calculated using the equation

K₁*=K₁κ₆/(κ₅+κ₆)

Inactivation of Inhibitor Cinn-GEE by Cysteamine. Cinn-GEE (1 mM) was incubated with 100 mM cysteamine hydrochloride (which had been neutralized by the addition of 1.0 equivalent of NaOH) in 100 μL of a 97:3 DMSO/H₂O mixture for 24 h. The treated inhibitor was then used in inhibition assays as described above. As a control, inhibition assay also performed with the same amount of cysteamine hydrochloride/NaOH solution.

NMR Spectroscopy of [¹³C]Cinn-GEE and [¹³C]Cinn-GEE-PTP1B Complex. All NMR experiments were performed on a Bruker DMX-600 NMR spectrometer equipped with a triple-resonance and 3-axes gradient probe at 300 K. The 2D ¹H-¹³C heteronuclear single-quantum coherence spectra (HSQC) were acquired using a constant-time sensitivity-enhanced method (35, 36). All samples were dissolved in a buffer containing 5 mM Hepes (pH 7.4), 150 mM NaCl, 5 mM EDTA, and 2 mM β-mercaptoethanol (94:6 H₂O:D₂O). The spectral widths were 9615 Hz in the ¹H dimension and 19621 Hz in the ¹³C dimension, with carrier frequencies at 4.7 and 150 ppm, respectively.

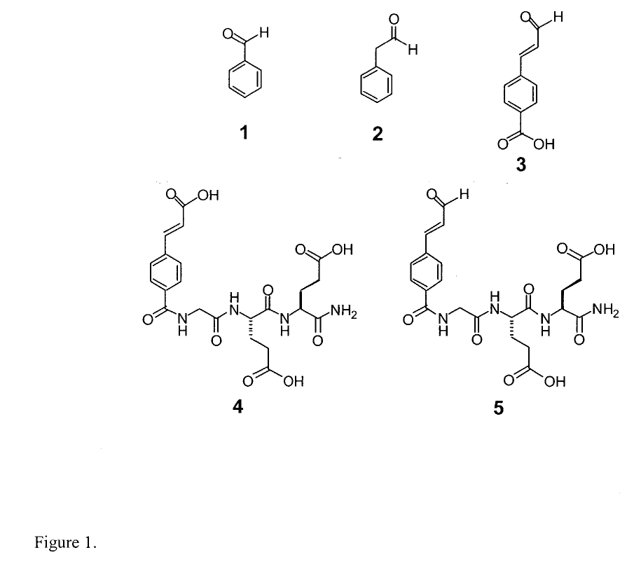

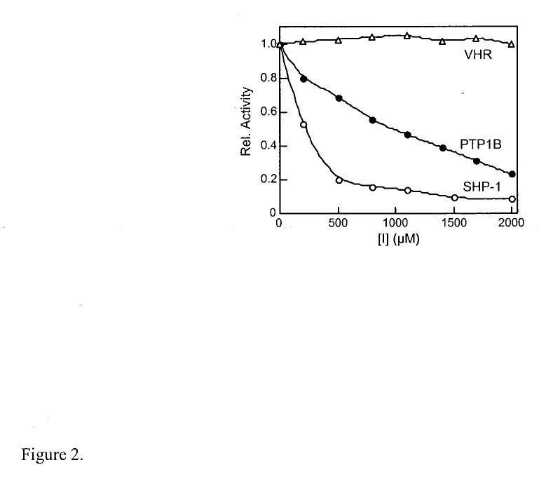

RESULTS Inhibition of PTPs by Simple Aldehydes. Peptidyl aldehydes represent an important class of inhibitors for cysteine proteases which, like PTPs, also utilize an active-site cysteine as the catalytic nucleophile (37). These aldehydes bind to the proteases through the formation of a covalent but reversible hemithioacetal adduct with the active-site cysteine. Calpeptin, a potent dipeptide aldehyde inhibitor of calpain, has recently been reported to show modest cross reactivity toward several PTPs (38, 39). These observations led us to hypothesize that aryl substituted aldehydes might serve as selective PTP inhibitors. We therefore tested several aryl aldehydes (FIG. 1) against tyrosine phosphatases PTP1B and SHP-1, and a dual-specificity phosphatase VHR. 4-Carboxycinnamaldehyde (3) was the most active against PTPs among the three aldehydes tested, with IC₅₀ values of 230 and 970 μM against SHP-1 and PTP1B, respectively (FIG. 2 and Table 1). It showed no significant inhibition against VHR up to 2 mM. Benzaldehyde was the least active inhibitor against all PTPs, presumably because its carbonyl group cannot readily reach the nucleophile(s) in the deep active-site pocket. All of the inhibitors were less active against VHR, which has a wider, shallower active site pocket as compared to PTPs (40). Due to limited solubility, the IC₅₀ values for some enzyme/inhibitor pairs could not be determined.

TABLE 1
Inhibitory Activity of Aldehydes against Various PTPs

| | Inhibition Constant (IC₅₀ or K_{I}*, μM) | | |
|---|---|---|---|
| Inhibitor | PTP1B | SHP-1 | VHR |
| 1 | NA | NA | NA |
| 2 | 15% (at 200 μM) | NA | NA |
| 3 | 970^{a} | 230^{a} | >2000^{a} |
| 4 | 0.079^{b} | | |
| 5 | 5.42 ± 0.94^{c} | 10.7 ± 1.4^{c} | 288 ± 112^{c} |

^{a}IC₅₀ values;
^{b}K_{I} value from Moran et al. (20);
^{c}K_{I}* values;
NA, no significant activity.

Slow-Binding Inhibition of PTPs by Cinn-GEE. The simple aldehydes presumably interact with only the active-site pocket of PTPs, limiting their inhibitory potency. One approach to improving their potency is to derivatize the aldehydes with functional groups that can interact with the protein surfaces near the active site. These additional interactions would also confer selectivity for a particular PTP on the inhibitor. As a proof of principle, we attached the tripeptide GEE to the para position of cinnamaldehyde to obtain N-[4-(3-oxo-1-propenyl)benzoyl]-Gly-Glu-Glu-NH₂ (Cinn-GEE) (compound 5 in FIG. 1). Cinn-GEE was synthesized from commercially available materials as detailed under Materials and Methods (Scheme 1).

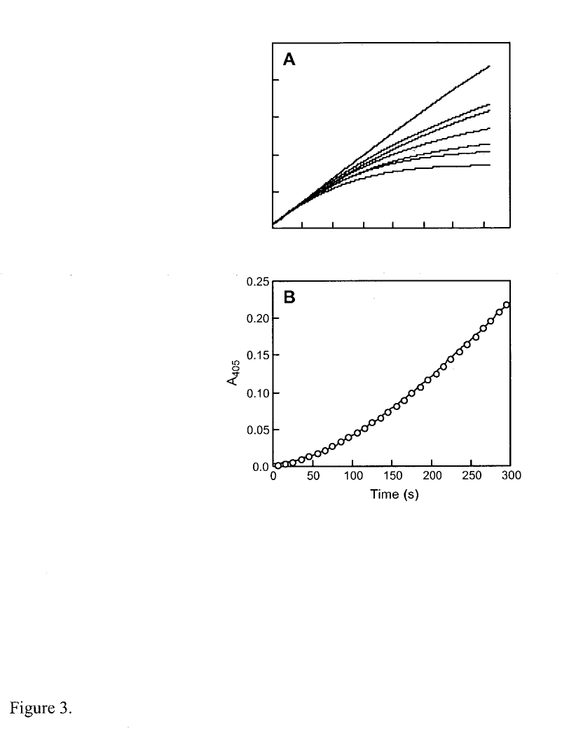

Cinn-GEE exhibited time-dependent inhibition toward PTP1B, SHP-1, and VHR. It resulted in biphasic curves when the hydrolysis of pNPP by PTPs was monitored in a continuous fashion, indicative of slow-binding inhibition (FIG. 3A) (41). The inhibition kinetics can be the following equation,

$$E + I {⇄}^{{K}_{1}} E \cdot I {{⇄}_{{k}_{6}}}^{{k}_{5}} E \cdot {I}^{*}$$

where K₁ is the equilibrium inhibition constant for the formation of the initial complex, E·I, and κ₅ and κ₆ the forward and reverse rate constants for the slow conversion of the initial E·I complex into a tight complex E·I*, respectively. The overall potency of the inhibitor is described by the overall equilibrium constant, K₁*=K₁κ₆/(κ₅+κ₆). The K₁* values of Cinn-GEE were 5.4, 10.7, and 288 μM against PTP1B, SHP-1, and VHR, respectively (Table 1). In order to distinguish the observed slow-binding behavior from time-dependent inactivation, PTP1B was pre-incubated with excess Cinn-GEE to form the E·I* complex, which was then rapidly diluted into an assay solution containing pNPP as substrate. The reaction progress as a function of time is shown in FIG. 3B. The slow reactivation of the enzyme with time is consistent with the slow-binding mechanism. Curve fitting indicates that the rate for reactivation of PTP1B (κ₆) is 0.56 min⁻¹. Based on the K₁ value of 550 μM for Cinn-GEE binding to PTP1B, the rate for the conversion of noncovalent E·I complex to the covalent E−I complex was determined as 56 min⁻¹. 4-Carboxycinnamaldehyde exhibited similar slow-binding behavior towards the PTPs (data not shown), although the K₁, κ₅, and κ₆ values could not be reliably determined due to the weak binding affinity. The tripeptide (GEE-NH₂) alone did not inhibit any of the enzymes.

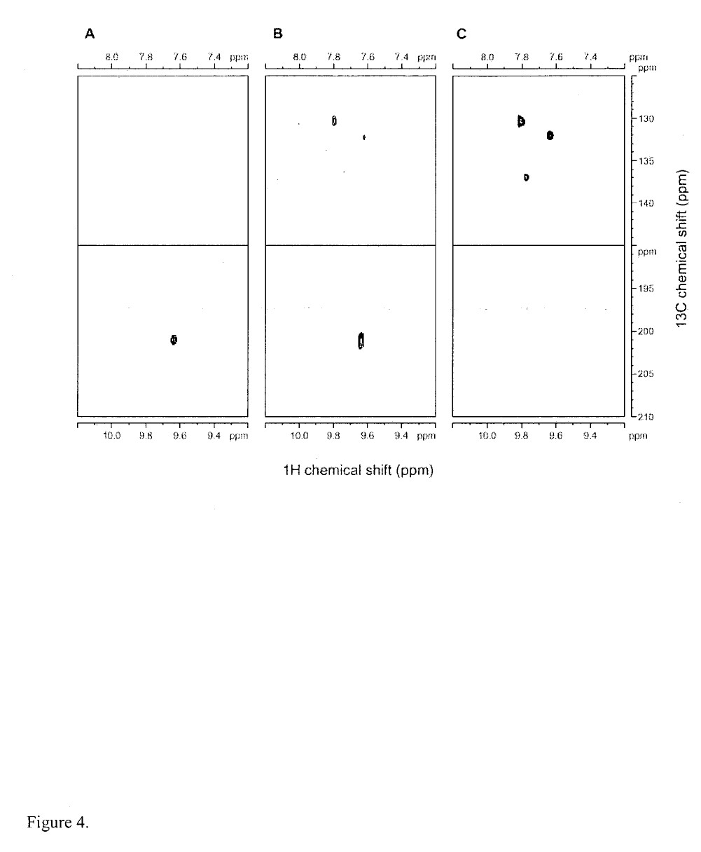

Mechanism of Inhibition. It was initially hypothesized that the E*I complex was the noncovalent complex of PTP1B and Cinn-GEE, whereas the E·I* represented a covalent adduct formed between the inhibitor aldehyde and the thiol group of the enzyme active-site cysteine. To test this hypothesis, we synthesized Cinn-GEE with ¹³C labeling at the aldehyde carbonyl carbon (Compound 14) and analyzed both the inhibitor and the enzyme-inhibitor complex by ¹H-¹³C HSQC NMR spectroscopy. When dissolved in the PTP assay buffer (pH 7.4), [¹³C]Cinn-GEE alone showed a single cross peak at δ 9.64 (¹H) and δ 201 (¹³C) in the NMR spectrum (FIG. 4A). This suggests that the free inhibitor existed predominantly in the aldehyde form in the aqueous solution. Upon the addition of increasing concentrations of PTP1B (with preincubation), the signal of the free inhibitor decreased, with concomitant appearance of two major peaks at δ 7.80/130 and δ 7.65/132, respectively, and a minor peak at δ 7.76/137 (FIG. 4B). Finally, when PTP1B was added in slight excess, the free inhibitor signal was completely converted into the above three peaks (FIG. 4C). No other signals were observed that could be attributed to either the free inhibitor or the enzyme-inhibitor complex. The same three peaks were also observed in the 1-D ¹³C NMR spectrum of the PTP1B/Cinn-GEE complex (data not shown). These unexpected results (chemical shift values and the appearance of multiple peaks) are inconsistent with the formation of a hemithioacetal. Instead, these observations are consistent with the formation of an enamine between the inhibitor aldehyde and the guanidine group of an arginine residue in the PTP active site.

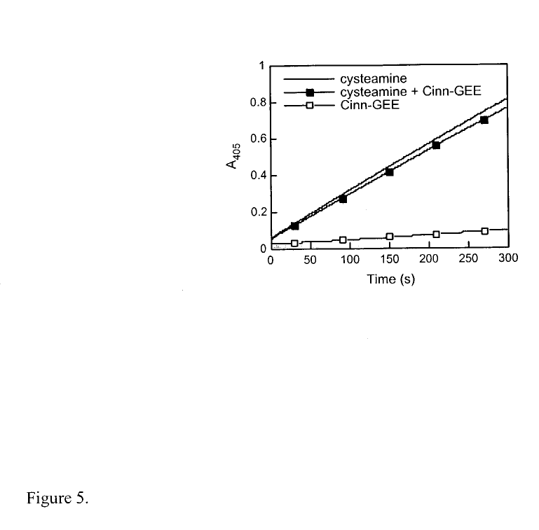

There is a slight possibility that the observed inhibitory activity of Cinn-GEE may be due to contamination by a small amount of the more potent cinnamic acid 4 (which could be formed through air oxidation of Cinn-GEE). This notion was tested by treating the inhibitor with excess cysteamine, which reacts with aldehydes to form a stable five-membered thiazolidine ring but should have no effect on the cinnamic acid inhibitor. As expected, prior treatment of Cinn-GEE with excess cysteamine abolished its inhibitory activity against PTP1B (FIG. 5). In addition, the cinnamic acid 4 was reported as a simple competitive inhibitor (20), whereas the observed inhibition in this work exhibited slow-binding characteristics, consistent with the formation of a reversible enzyme-inhibitor adduct. Taken together, these data rule out the possibility that the observed inhibition was caused by cinnamic acid contamination.

In accordance with the methods of the present invention, a different approach to PTP inhibitor design has been used. The strategy was to covalently modify the conserved active-site residues of PTPs using hydrophobic core structures (29). Because the catalytic cysteine is conserved among all PTPs and is exceptionally acidic (e.g., pKa=4.7 in Yersinia PTP) (41), it was reasoned that a peptidyl aldehyde might selectively inhibit PTPs by forming a hemithioacetal adduct with the cysteine thiolate. Since aryl aldehydes are only marginally soluble in water, they should have relatively low desolvation energy, a property that should make them more membrane permeable as well as promote binding to an enzyme active site. In this work, it is shown that certain aryl-substituted aldehydes indeed act as reversible inhibitors of PTPs. As one might expect from their small sizes, the simple aldehydes do not have high affinity to the PTP active site (IC₅₀ values in the high μM to mM range). However, attachment of a tripeptide GEE to the para position of cinnamaldehyde resulted in an inhibitor of substantially improved affinity to both PTP1B (K₁*=5.4 μM) and SHP-1 (K₁*=10.7 μM). This suggests that by attaching a properly designed PTP recognition motif or by screening a combinatorial library, one should be able to obtain highly potent and specific inhibitors against a PTP of interest.

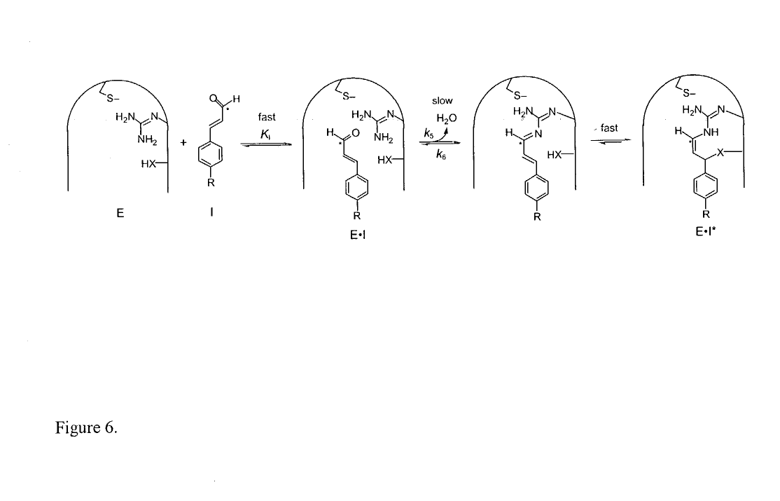

Cinn-GEE behaves as a slow-binding inhibitor. Since simple aldehydes (e.g., 4-carboxycinnamaldehyde) exhibit similar slow-binding inhibition, the E·I to E·I* conversion is likely due to structural changes within the active site. Initially, it appeared that the slow-binding behavior could be explained by the time-dependent formation of a reversible hemithioacetal adduct between the aldehyde and the active-site cysteine. Such a mechanism is commonly observed for inhibition of cysteine proteases by peptidyl aldehydes (42, 43). Surprisingly, ¹H-13C HSQC experiments with ¹³C-labeled Cinn-GEE and PTP1B showed three cross peaks at δ 7.80/130, δ 7.65/132, and δ7.76/137 (FIG. 4). These results are inconsistent with the formation of a hemithioacetal, as a hemithioacetal would show two or less cross peaks at δ ˜5.0 (for the aldehyde-derived ¹H) and δ ˜85 (¹³C) (44-46). They also rule out the possibility of free aldehyde (δ9.64/201) or the hydrate form (which should have a single cross peak at δ ˜5.2/˜95) as the bound inhibitor form in the PTP active site. We propose that the bound inhibitor form an enamine structure with the guanidine group of Arg-221 (FIG. 6). Arg-221 is a universally conserved residue present in the PTP signature motif, HCxxGxxR(S/T). It is critical for pY substrate binding and transition-state stabilization by forming a pair of hydrogen bonds between the phosphate non-bridging oxygen atoms and the Nε and Nη of Arg-221 (47). Binding of the cinnamaldehyde derivatives likely places the carbonyl group at a similar position as the phosphate of a substrate. The close proximity between the aldehyde and the guanidinium group presumably drives the deprotonation of the guanidinium group and the formation of a conjugated imine intermediate. Conjugate addition at the benzylic position by a yet unidentified nucleophile produces the enamine structure (FIG. 5). This unknown nucleophile may be either a protein side chain (e.g., the second amino group of Arg-221 or Asp-181 which is the general acid/base during catalysis) or a water molecule. This working model provides a sensible explanation for all of the experimental observations. Since the formation of imine/enamine is readily reversible, it explains the reversible nature of the inhibitors. Because imine formation requires the expulsion of a water molecule from a relatively deep active site, the reaction is expected to be slow, as is observed. The unknown nucleophile may add from either side of the plane and the double bonds in the enamine structure may assume either cis or trans configuration. This can account for the formation of multiple products in the HSQC spectrum. Finally, the observed chemical shift values (δ ˜132) suggest that the carbon atom is sp2 hybridized (in the form of imine or enamine). The actual δ values are more consistent with the enamine structure, as the ¹³C resonance of a typical imine is at δ 150-160. Theoretical calculations using ACD/CNMR software (from Advanced Chemistry Development, Inc., Toronto, Canada) predicted that the aldehyde-derived carbon atom in the imine and enamine model systems formed from cinnamaldehyde and guanidine has ¹³C resonance at δ 162 and 141, respectively. Since we failed to observe the imine intermediate under any conditions, we propose that the conjugate addition by group X is a rapid step.

While surprising, in retrospect, the lack of reaction between the aldehyde group and the active-site cysteine of PTPs makes sense. A nucleophile must approach from above or below the carbonyl plane in order to have a successful addition reaction. Peptidyl aldehyde inhibitors of cysteine proteases are so designed that the aldehyde carbonyl occupies the same position as the amide carbonyl of a scissile bond. Thus, in the E·I complex, the cysteine thiol is properly positioned for nucleophilic attack on the aldehyde. In PTPs, the thiolate is situated at the bottom of the active-site pocket (47). This position is ideally suited for a back attack on an incoming phosphate group, with a co-linear S—P—OPh bond angle in the transition state. However, when cinnamaldehyde binds to the PTP active site, the thiolate is likely face the edge of the carbonyl plane. This geometric restriction presumably prevents a successful nucleophilic attack. On the other hand, Arg-221 side chain is on the wall of the active-site pocket (47). This position permits a side attack on the carbonyl plane.

Attachment of a tripeptide Gly-Glu-Glu to the para position of cinnamaldehyde resulted in an inhibitor (Cinn-GEE) of substantially increased potency against all three enzymes studied (e.g., K₁*=5.4 μM against PTP1B). The mechanism of inhibition was investigated using Cinn-GEE specifically labeled with 13C at the aldehyde carbon and ¹H-¹³C heteronuclear single-quantum coherence spectroscopy. While Cinn-GEE alone showed a single cross peak at δ 9.64 (¹H) and δ 201 (¹³C), the PTP1B/Cinn-GEE complex showed three distinct cross peaks at δ 7.6-7.8 (¹H) and 130-137 (¹³C). This rules out the possibility of a free aldehyde, aldehyde hydrate, or hemithioacetal as the enzyme bound inhibitor form. Instead, the data are consistent with the formation of an enamine between the aldehyde group of the inhibitor and the guanidine group of a conserved arginine residue in the PTP active site (Arg-221 in PTP1B).

In summary, aryl-substituted aldehydes act as slow-binding inhibitors of PTPs. The time-dependent inhibition is most likely due to the formation a reversible adduct between the inhibitor and the conserved active-site arginine. These aryl aldehydes and ketones should provide a general core structure that can be further developed into highly potent and specific inhibitors against PTPs. The additional experiments below provide additional pY mimetics that work with the methods provided herein.

### Example 2

### Simple Aldehydes that have been Tested Against PTPs

All of the following aldehydes have been tested against phosphatases PTP1B and/or SHP-1 (the catalytic domain). Unless otherwise noted, the values represent inhibition of PTP1B, the prototypical protein tyrosine phosphatase. The compounds in boxes are synthesized through methods that have appeared in the literature. The rest are commercially available. The conclusion is that many aryl-substituted aldehydes show weak to modest inhibitory activity toward PTPs. The cinnamaldehydes appear to be particularly effective.

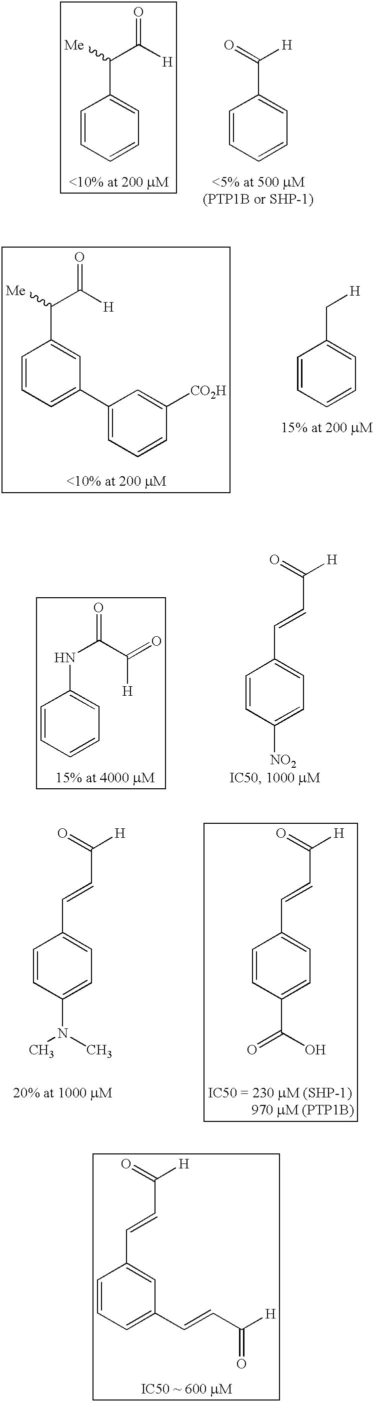

### Example 3

### Peptidyl Aldehydes that have been Synthesized and Tested Against PTPs

The first compound was a result of rational design, based on the earlier finding that a tripeptide GEE can enhance binding of cinnamic acid to PTP1B (20). All other compounds were identified from combinatorial libraries constructed in this lab. They were then individually synthesized on larger scales and tested against various PTPs. Unless otherwise noted, the inhibition constants are against PTP1B.

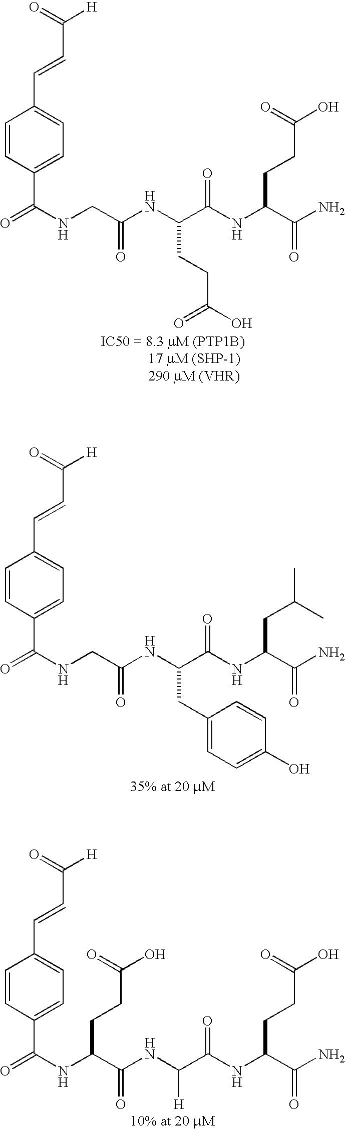

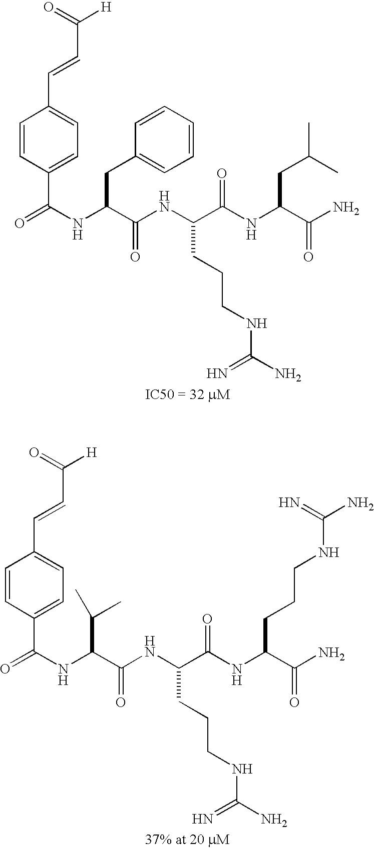

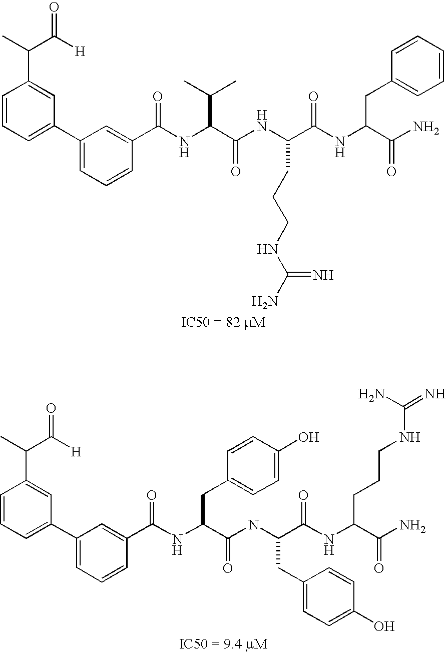

### Example 4

### Peptidyl Aldehydes that have been Selected from Combinatorial Libraries—Library 1

The following compounds were selected from combinatorial libraries against PTP1B and SHP-1 catalytic domain. The fact that they are selected from the diverse libraries suggests that they should all be fairly potent inhibitors of the PTP used in the screenings. The following are selected as inhibitors for PTP1B, SHP-1, and SH2 domains.

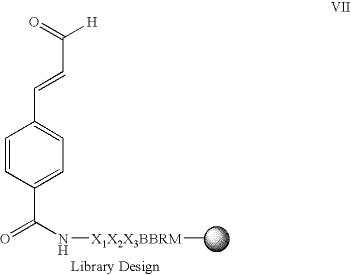

Selected Inhibitors for PTP1B (X₁X₂X₃=19 α-amino acid except for cysteine):

| | | | | | | |
|---|---|---|---|---|---|---|
| PVL | NSV | QLL | FPS | NIY | AAF | NLG |
| APL | PQH | PQL | MLF | EVM | YYT | QMP |
| APP | NAS | GFQ | ILE | TPH | FEA | YIF |
| VRR | LRF | DVK | NPH | RKR | VRK | GLW |
| FRL | RFR | LRL | GNR | FRG | GRL | LTR |
| YRK | SYR | RRL | GRR | SRF | LYR | YNR |
| RTR | TLR | GYY | LRF | NFW | TRL | YRL |
| RFL | FRR | RGR | RFY | NRF | GLR | DRR |
| NRF | RLR | RVF | ARR | FRL | DRR | TRR |
| RVR | RRK | FRL | QLR | RNV | NPW | FRL |
| FPR | GRF | SKR. | | | | |

Selected Inhibitors for SHP-1 Catalytic Domain (X₁X₂X₃=17 α-amino acid except for cysteine, arginine, and lysine):

| | | | | | | |
|---|---|---|---|---|---|---|
| YWY | INE | VSH | LPL | VLY | VDH | DHG |
| LLF | LDE | EDM | VLE | DTA | VSN | QGE |
| SME | FVQ | PAL | QDS | NTL | EAY | FML |
| IIH | VYN | NFI | VPG | GDV | HQE. | |

Selected Inhibitors for SHP-1 N-SH2 Domain (X₁X₂X₃=19 α-amino acid except for cysteine):

| | | | | | | |
|---|---|---|---|---|---|---|
| VHL | YTR | DRN | RLQ | EEY | NDS | RGR |
| RML. | | | | | | |

### Example 5

### Peptidyl Aldehydes that have been Selected from Combinatorial Libraries—Library 2

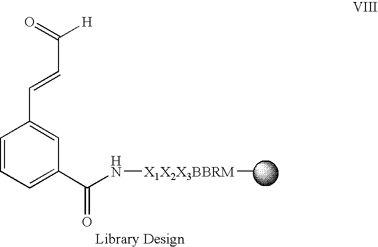

Selected Inhibitors for PTP1B (X₁X₂X₃=17 α-amino acid except for Cys, Arg, and Lys):

| | | | | | | |
|---|---|---|---|---|---|---|
| QTQ | EGP | IHV | YNH | QVT | GVN | PVY |
| PFL. | | | | | | |

Selected Inhibitors for SHP-1 Catalytic Domain (X₁X₂X₃=17 α-amino acid except for Cys, Arg, and Lys):

| | | | | | | |
|---|---|---|---|---|---|---|
| DGL | AYV | EVA | VDL | TYG | SII | LED |
| QAL | QYP | VTI | MMM. | | | |

Selected Inhibitors for SHP-1 N-SH2 Domain (X₁X₂X₃=19 α-amino acid except for Cys):

| | | | | | | |
|---|---|---|---|---|---|---|
| ARL | RWL | ARN | GRT | RRV | VAR | PLL |
| IAH | NSR | IKL | LRR | DVR | IEF | EYR |
| IRF | VKR. | | | | | |

### Example 6

### Peptidyl Aldehydes that have been Selected from Combinatorial Libraries—Library 3

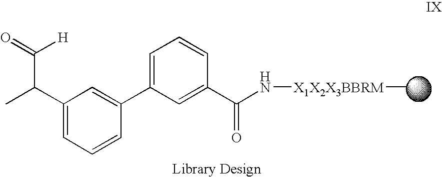

Selected Inhibitors for PTP1B (X₁X₂X₃=19 α-amino acid except for cysteine):

| | | | | | | |
|---|---|---|---|---|---|---|
| YRY | VDW | RWR | VWA | VAR | DKA | GGA |
| DFL | LYM | YPY | YRL | VRM | VRF | LKW |
| IRF | RSF | WFL | RGR | EGA | EFP | YYR |
| WKV | VAW | WLR | VLL | YYR | NHY | SFW |
| YPL | RRA | YSP | FVG | ALG | SWA | GGA |
| GFN | FEY | ENV | MLM | NVS | VYM | YSL |
| AEN | EHL | LVY | VEM | VYT | GPT | GTE. |

Selected Inhibitors for SHP-1 Catalytic Domain (X₁X₂X₃=17 α-amino acid except for cysteine, arginine, and lysine):

| | | | | | | |
|---|---|---|---|---|---|---|
| SYF | VLF | VLV | QPF | YPA | AVA | IGP |
| HHA | SYP | FGA | IVT | QVS | QLV | TFH |
| GQY | YMI | VVS. | | | | |

Selected Inhibitors for SHP-1 N-SH2 (X₁X₂X₃=19 α-amino acid except for cysteine):

| | | | | | | |
|---|---|---|---|---|---|---|
| EDY | RTH | EHV | NYP | VVT | HIH | EVF |
| KQI | ILR | PYY | HRM | SQY | KVR | LHF |
| VHV. | | | | | | |

### Examples 7-11

### Additional PTP inhibitors

Example 7

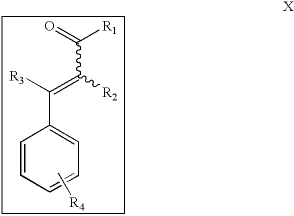

wherein R₁=H, alkyl (C1-C7), benzyl, or alkoxymethyl (ROCH2-, R═C1-C7);

R₂, R₃═H, halogen (F, Cl, Br, I), alkyl (C1-C7), or alkoxy RO— (C1-C7);

R₄—H, halogen (F, Cl, Br, I), —NO2 (nitro), —CN (cyano), R—, phenyl, RO—, RCO—, ROCO—, RCOO—, RNHCO—, RCONH—, or peptidyl. R may be H or alkyl (C1-C20). R₄ may be either mono- or multiple-substitution at ortho-, meta-, and/or para-positions.

### Example 8

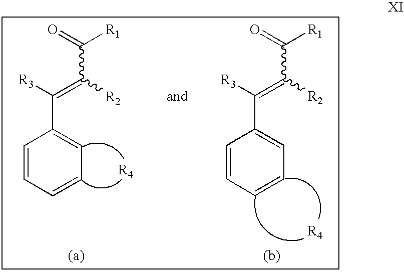

Wherein R₁═H, alkyl (C₁-C₇), benzyl, or alkoxymethyl (ROCH₂—, R═C₁-C₇);

R₂, R₃═H, halogen (F, Cl, Br, I), alkyl (C₁-C₇), or alkoxy RO—(C₁-C₇);

R₄=fused aromatic rings (with or without heteroatoms) or other saturated or partially saturated carbocycles and heterocycles.

### Example 9

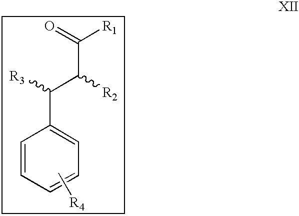

Wherein R₁═H, alkyl (C₁-C₇), benzyl, or alkoxymethyl (ROCH₂—, R═C₁-C₇);

R₂, R₃═H, halogen (F, Cl, Br, I), alkyl (C₁-C₇), or alkoxy RO—(C₁-C₇);

R₄═H, halogen (F, Cl, Br, I), —NO2 (nitro), —CN (cyano), R—, phenyl, RO—, RCO—, ROCO—, RCOO—, RNHCO—, RCONH—, or peptidyl. R may be H or alkyl (C₁-C₂₀). R₄ may be either mono- or multiple-substitution at ortho-, meta-, and/or para-positions.

### Example 10

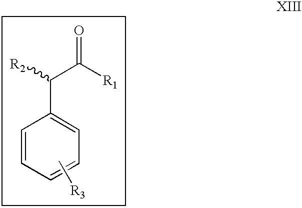

Wherein R₁═H, alkyl (C₁-C₇), benzyl, or alkoxymethyl (ROCH₂—, R═C₁-C₇);

R₂═H, halogen (F, Cl, Br, I), alkyl (C₁-C₇), or alkoxy RO—(C₁-C₇);

R₃═H, halogen (F, Cl, Br, I), —NO2 (nitro), —CN (cyano), R—, phenyl, RO—, RCO—, ROCO—, RCOO—, RNHCO—, RCONH—, or peptidyl. R may be H or alkyl (C₁-C₂₀). R₃ may be either mono- or multiple-substitution at ortho-, meta-, and/or para-positions. R₃ may also be fused carbocycles or heterocycles.

### Example 11

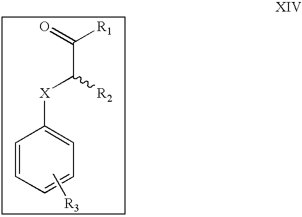

Wherein R₁═H, alkyl (C₁-C₇), benzyl, or alkoxymethyl (ROCH₂—, R═C₁-C₇);

R₂═H, halogen (F, Cl, Br, I), alkyl (C₁-C₇), or alkoxy RO- (C₁-C₇);

X═O, S, or NH;

R₃═H, halogen (F, Cl, Br, I), —NO2 (nitro), —CN (cyano), R—, phenyl, RO—, RCO—, ROCO—, RCOO—, RNHCO—, RCONH—, or peptidyl. R may be H or alkyl (C₁-C₂₀). R₃ may be either mono- or multiple-substitution at ortho-, meta-, and/or para-positions. R₃ may also be fused carbocycles or heterocycles.

The present invention comprises a method of treating disorders requiring regulation of specific PTPs, including pharmaceutical compositions comprising pY mimetics. The pharmaceutical composition comprises a therapeutically effective amount of a compound of formula I-IX, or a derivative or pharmaceutically acceptable salt thereof, in association with at least one pharmaceutically acceptable carrier, adjuvant, or diluent (collectively referred to herein as “carrier materials”) and, if desired, other active ingredients. The active compounds of the present invention may be administered by any suitable route known to those skilled in the art, preferably in the form of a pharmaceutical composition adapted to such a route, and in a dose effective for the treatment intended. The active compounds and composition may, for example, be administered orally or intra-vascularly.

The administration of the present invention may be for either prevention or treatment purposes. The methods and compositions used herein may be used alone or in conjunction with additional therapies known to those skilled in the art in the prevention or treatment of disorders which are caused by PTPs, whether by mutation of the PTP, as in type II diabetes, or which are treated by suppressing the function of the PTPs, as to prevent rejection in transplant recipients. Alternatively, the methods and compositions described herein may be used as adjunct therapy.

The phrase “adjunct therapy” (or “combination therapy”), in defining use of a compound of the present invention and one or more other pharmaceutical agent, is intended to embrace administration of each agent in a sequential manner in a regimen that will provide beneficial effects of the drug combination, and is intended as well to embrace co-administration of these agents in a substantially simultaneous manner, such as in a single formulation having a fixed ratio of these active agents, or in multiple, separate formulations for each agent.

For oral administration, the pharmaceutical composition may be in the form of, for example, a tablet, capsule, suspension or liquid. The pharmaceutical composition is preferably made in the form of a dosage unit containing a particular amount of the active ingredient. Examples of such dosage units are capsules, tablets, powders, granules or a suspension, with conventional additives such as lactose, mannitol, corn starch or potato starch; with binders such as crystalline cellulose, cellulose derivatives, acacia, corn starch or gelatins; with disintegrators such as corn starch, potato starch or sodium carboxymethyl-cellulose; and with lubricants such as talc or magnesium stearate. The active ingredient may also be administered by injection as a composition wherein, for example, saline, dextrose or water may be used as a suitable carrier.

For intravenous administration, the compound may be combined with a sterile aqueous solution which is preferably isotonic with the blood of the recipient. Such formulations may be prepared by dissolving solid active ingredient in water containing physiologically compatible substances such as sodium chloride, glycine, and the like, and having a buffered pH compatible with physiological conditions to produce an aqueous solution, and rendering said solution sterile. The formulations may be present in unit or multi-dose containers such as sealed ampoules or vials.

The dosage form and amount can be readily established by reference to known treatment or prophylactic regiments. The amount of therapeutically active compound that is administered and the dosage regimen for treating a disease condition with the compounds and/or compositions of this invention depends on a variety of factors, including the age, weight, sex, and medical condition of the subject, the severity of the disease, the route and frequency of administration, and the particular compound employed, as well as the pharmacokinetic properties of the individual treated, and thus may vary widely. Such treatments may be administered as often as necessary and for the period of time judged necessary by the treating physician. One of skill in the art will appreciate that the dosage regime or therapeutically effective amount of the inhibitor to be administrated may need to be optimized for each individual. The pharmaceutical compositions may contain active ingredient in the range of about 0.1 to 2000 mg, preferably in the range of about 0.5 to 500 mg and most preferably between about 1 and 200 mg. A daily dose of about 0.01 to 100 mg/kg body weight, preferably between about 0.1 and about 50 mg/kg body weight, may be appropriate. The daily dose can be administered in one to four doses per day.

All documents referenced herein are incorporated by reference.

Although this invention has been described with respect to specific embodiments, the details of these embodiments are not to be construed as limitations.

### References

1. Neel, B. G., & Tonks, N. K. (1997) Curr. Opin. Cell Biol. 9, 193-204.

2. Hunter, T. (2000) Cell 100, 113-127.

3. Zhang, Z. -Y. (2001) Curr. Opin. Chem. Biol. 5, 416-423.

4. Elchelby, M., Payette, P., Michaliszyn, E., Cromlish, W., Collins, S., Lee Loy, A., Normandin, D., Cheng, A., Himms-Hagen, J., Chan, C. -C., Ramanchandran, C., Gresser, M. J., Tremblay, M. L., Kennedy, B. P. (1999) Science 283, 1544-1548.

5. Burke Jr., T. R., & Zhang, Z. -Y. (1998) Biopolymers 47, 225-241.

6. Burke Jr., T. R., Kole, H. K., & Roller, P. P. (1994) Biochem. Biophys. Res. Commun. 204, 129-134.

7. Taylor, W. P., Zhang, Z. -Y., & Widlanski, T. S. (1996) Bioorg. Med. Chem. 4, 1515-1520.

8. Taylor, S. D., Kotoris, C. C., Dinaut, A. N., Wang, Q., Ramachandran, C., & Huang, Z. (1998) Bioorg. Med. Chem. 6, 1457-1468.

9. Taing, M., Keng, Y. -F., Shen, K., Wu, L., Lawrence, D. S., & Zhang, Z. -Y. (1999) Biochemistry 38, 3793-3803.

10. Shen, K., Keng, Y. -F., Wu, L., Guo, X. -L., Lawrence, D. S., & Zhang, Z. -Y. (2001) J. Biol. Chem. 276,47311-47319.

11. Ye, B., Akamatsu, M., Shoelson, S. E., Wolf, G., Giogetti-Peraldi, S., Yan, X., Roller, P. P., & Burke, T. R. Jr. (1995) J. Med. Chem. 38, 4270-4275.

12. Burke Jr., T. R., Ye, B., Akamatsu, M., Ford, H., Yan, X. J., Kole, H. K., Wolf, G., Shoelson, S. E., & Roller, P. P. (1996) J. Med. Chem. 39, 1021-1027.

13. Akamatsu M, Roller P P, Chen L, Zhang Z -Y, Ye B, & Burke Jr, T R. (1997) Bioorg. Med. Chem. 5, 157-163.

14. Roller, P. P., Wu, L., Zhang, Z. -Y., & Burke Jr., T. R. (1998) Bioorg. Med. Chem. Lett. 8, 2149-2150.

15. Sarmiento, M., Wu, L., Keng, Y. -Y., Song, L., Luo, Z., Huang, Z., Wu, G. -Z., Yuan, A. K., & Zhang, Z. -Y. (2000) J. Med. Chem. 43, 146-155.

16. Iversen, L. F., Andersen, H. S., Branner, S., Mortensen, S. B., Peters, G. H., Norris, K., Olsen, O. H., Jeppesen, C. B., Lundt, B. F., Ripka, W., Møller, K. B., & Møller, N. P. H. (2000) J. Biol. Chem. 275: 10300-10307.

17. Wrobel, J., Li, Z., Sredy, J., Sawicki, D. R., Seestaller, L., & Sullivan, D. (2000) Bioorg. Med. Chem. Lett. 10, 1535-1538.

18. Chen, Y. T., Onaran, M. B., Doss, C. J., & Seto, C. T. (2001) Bioorg. Med. Chem. Lett. 11, 1935-1938.

19. Malamas, M. S., Sredy, J., Moxham, C., Katz, A., Xu, W. X., McDevitt, R., Adebayo, F. O., Sawicki, D. R., Seestaller, L., Sullivan, D., & Taylor, J. R. (2000) J. Med. Chem. 43, 1293-1310.

20. Moran, E. J., Sarshr, S., Cargill, J. E., Shahbaz, M. M., Lio, A., Mjalli, A. M. M., & Annstrong, R. W. (1995) J. Am. Chem. Soc. 117, 10787-10788.

21. Cao, X., Moran, E. J., Siev, D., Lio, A., Ohashi, C., & Mjalli, A. M. M. (1995) Bioorg. Med. Chem. Lett. 5, 2953-2958.

22. Umezawa, K. (1995) Adv. Enzyme Regul. 35, 43-53.

23. Miski, M., Shen, X., Cooper, R., Gillum, A. M., Fisher, D. K., Miller, R. W., & Higgins, T. J. (1995) Bioorg. Med. Chem. Lett. 5, 1519-1522.

24. Zhang, Y. -L., Keng, Y. -F., Zhao, Y., Wu. L., & Zhang, Z. -Y. (1998) J. Biol. Chem. 273, 12281-12287.

25. Zhang, Z. -Y. & Dixon, J. E. (1994) Advance in Enzymology 68, 1-36.

26. Zhang, Z. -Y., Wang, Y., Wu, L., Fauman. E. B., Stuckey, J. A., Schubert, H. L., Saper, M. A., & Dixon, J. E. (1994) Biochemistry 33, 15266-15270.

27. Guan, K. L., & Dixon, J. E. (1990) Science 249, 553-556.

28. Pot, D. A., & Dixon, J. E. (1992) J. Biol. Chem. 267, 140-143.

29. Arabaci, G., Guo, X. -C., Beebe, K. D., Coggeshall, K. M., & Pei, D. (1999) J. Am. Chem. Soc. 121, 5085-5086.

30. Pei, D., Neel, B. G., & Walsh, C. T. (1993) Proc. Natl. Acad. Sci. U.S.A. 91, 1092-1096.

31. Puius, Y. A., Zhao, Y., Sullivan, M., Lawrence, D. S., Almo, S. C., Zhang, Z. -Y. (1997) Proc. Natl. Acad. Sci. U.S.A. 94, 13420-13425.

32. Denu, J. M., Zhou, G., Wu, L., Zhao, R., Yuvaniyama, J., Saper, M. A., Dixon, J. E. (1995) J. Biol. Chem. 270, 3796-3803.

33. Daubresse, N., Francesch, C., Rolando, C. (1998) Tetrahedron 54, 10761-10770.

34. Lu, T. -J., Yang, J. -F., Shen, L. -J. (1995) J. Org. Chem. 60, 2931-2934.

35. Vuister, G. W., & Bax, A. (1992) J. Magnetic Resonance 98, 428-435.

36. Schedletzky, O., Glaser, S. J., Sorensen, O. W., & Griesinger, C. (1994) J. Biomol. NMR 4, 301-306.

37. Veber, D. F., Thompson, S. K. (2000) Curr. Opin. Drug Discovery Dev. 3, 362-369.

38. Schoenwaelder, S. M., and Burridge, K. (1999) J. Biol. Chem. 274, 14359-14367.

39. Schoenwaelder, S. M., Petch, L. A., Williamson, D., Shen, R., Feng, G. -S., and Burridge, K. (2000) Curr. Biol. 10, 1523 1526.

40. Yuvaniyama, J.; Denu, J. M.; Dixon, J. E.; Saper, M. A. (1996) Science 272, 1328-1331.

41. Morrison, J. F., & Walsh, C. T. (1988) Adv. Enzymol. 61, 201-301.

42. Zhang, Z. -Y., & Dixon, J. E. (1993) Biochemistry 32, 9340-9345.

43. Lewis, C. A. Jr., & Wolfenden, R. (1977) Biochemistry 16, 4890-4894.

44. Bendall, M. R., Cartwright, I. L., Clark, P. I., Lowe, G., & Nurse, D. (1977) Eur. J. Biochem. 79, 201-209.

45. Zhou, M., & Van Etten, R. L. (1999) Biochemistry 38, 2636-2646.

46. Sundaramoorthi, R., Siedem, C., Vu, C. B., Dalgarno, D. C., Laird, E. C., Botfield, M. C., Combs, A. B., Adams, S. E., Yuan, R. W., Weigele, M., & Narula, S. S. (2001) Bioorg. Med. Chem. Lett. 11, 1665-1669.

47. Jia, Z., Barford, D., Flint, A. J., & Tonks, N. K. (1995) Science 268, 1754-1758.

48. Urbanek, R. A., Suchard, S. J., Steelman, G. B., Knappenberger, K. S., Sygowski, L. A., Veale, C. A., & Chapdelaine, M. J. (2001) J. Med. Chem. 44, 1777-1793.

The invention claimed is:

1. A method of reversibly inhibiting protein tyrosine phosphatases (PTPs), the PTPs having a binding site comprising an arginine-containing active site and a reactive surface near the active site, the method comprising introducing a neutral phosphotyrosine (pY) mimetic to the PTP to be inhibited, the pY mimetic comprising a reversibly binding molecule, the molecule comprising:
a. a first reactive group that forms a reversible imine or enamine adduct with the active site arginine;
b. an aryl group, wherein the aryl group provides the molecule with affinity to the active site; and
c. a second reactive group that interacts with the reactive surface near the active site through molecular interactions, wherein the molecular interactions comprise one or more of electrostatic interactions, hydrogen-bonding interactions, hydrophobic interactions, and van der Waals interactions.

2. The method of claim 1 wherein the first reactive group is selected from aldehydes and mono-ketones.

3. The method of claim 1 wherein the PTP is selected from the group consisting of PTP1B, SHP-1, VHR, and CD45.

4. The method of claim 3 wherein the PTP is PTP1B.

5. The method of claim 1 wherein the neutral phosphotyrosine mimetic is a compound of Formula I or a pharmaceutically acceptable salt thereof, wherein Formula I is: 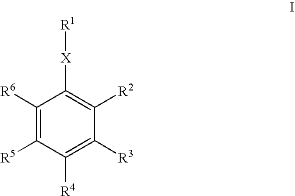
wherein
X is selected from the group consisting of C, N, O, and S;
provided when X is N or C, X may be further substituted with a substituent selected from the group consisting of H, halo, C₁-C₇ alkyl, aryl, alkylaryl, and C₁-C₇ alkoxy;
R¹ is selected from the group consisting of C₁-C₇ aldehyde and C₁-C₇ ketone; and R₁ is optionally substituted at any substitutable position with H, halo, aryl, alkylaryl, C₁-C₇ alkyl, C₁-C₇ haloalkyl, or C₁-C₇ alkoxy;
R²-R⁶ are selected from the group consisting of H, F, Cl, Br, I, NO₂, CN, OH, C₁-C₂₀ alkyl, C₁-C₂₀ alkenyl, aryl, alkylaryl, arylalkyl, C₁-C₂₀ alkoxy, R⁷OCO—, R⁷COO—, R⁷NHCO—, R⁷CONH—, peptidyl, arylpeptidyl and combinations thereof;
wherein R⁷ is selected from the group consisting of H, C₁-C₂₀ alkyl, C₁-C₂₀ alkenyl, aryl, alkylaryl, arylalkyl, and C₁-C₂₀ carboxylic acids; and
optionally, two of R²-R⁶ on adjacent C atoms may be joined to form a ring structure, wherein a fused polycyclic structure will be formed, the ring structure selected from the group consisting of aromatic, heterocyclic aromatic, saturated carbocyclic, saturated heterocyclic, partially saturated carbocyclic, and partially saturated heterocyclic.

6. The method of claim 1 wherein the phosphotyrosine mimetic is a compound of Formula II or a pharmaceutically acceptable salt thereof, wherein Formula II is: 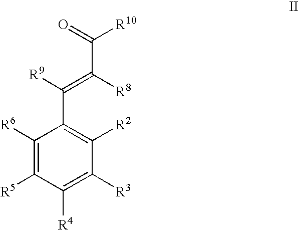
wherein
R²-R⁶ are selected from the group consisting of H, F, Cl, Br, I, NO₂, CN, OH, C₁-C₂₀ alkyl, C₁-C₂₀ alkenyl, aryl, alkylaryl, arylalkyl, C₁-C₂₀ alkoxy, R⁷OCO—, R⁷COO—, R⁷NHCO—, R⁷CONH—, peptidyl, arylpeptidyl and combinations thereof;
wherein R⁷ is selected from the group consisting of H, C₁-C₂₀ alkyl, C₁-C₂₀ alkenyl, aryl, alkylaryl, arylalkyl, and C₁-C₂₀ carboxylic acids;
R⁸ and R⁹ are selected from the group consisting of H, halo, C₁-C₇ alkyl, and C₁-C₇ alkoxy; and
R¹⁰ is selected from the group consisting of H, C₁-C₇ alkyl, benzyl, C₁-C₇ haloalkyl, and C₁-C₇ alkoxymethyl.

7. The method of claim 1 wherein the phosphotyrosine mimetic is a compound of Formula III or a pharmaceutically acceptable salt thereof, wherein Formula III is: 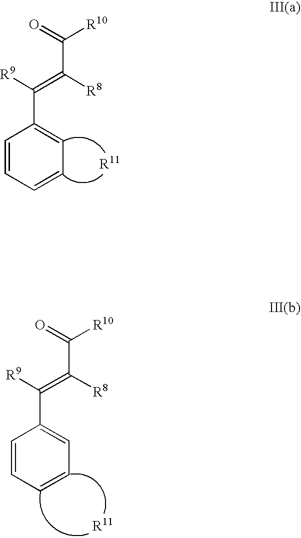
wherein
R⁸ and R⁹ are selected from the group consisting of H, halo, C₁-C₇ alkyl, and C₁-C₇ alkoxy;
R¹⁰ is selected from the group consisting of H, C₁-C₇ alkyl, benzyl, C₁-C₇ haloalkyl, and C₁-C₇ alkoxymethyl; and
R¹¹ is a fused ring selected from the group consisting of aromatic, heterocyclic aromatic, saturated carbocyclic, saturated heterocyclic, partially saturated carbocyclic, and partially saturated heterocyclic.

8. The method of claim 1 wherein the phosphotyrosine mimetic is a compound of Formula IV or a pharmaceutically acceptable salt thereof, wherein Formula IV is: 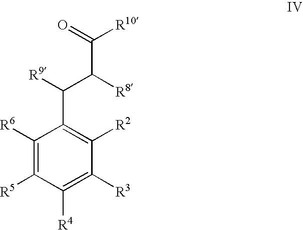
wherein
R²-R⁶ are selected from the group consisting of H, F, Cl, Br, I, NO₂, CN, OH, C₁-C₂₀ alkyl, C₁-C₂₀ alkenyl, aryl, alkylaryl, arylalkyl, C₁-C₂₀ alkoxy, R⁷OCO—, R⁷COO—, R⁷NHCO—, R⁷CONH—, peptidyl, arylpeptidyl, and combinations thereof;
wherein R⁷ is selected from the group consisting of H, C₁-C₂₀ alkyl, C₁-C₂₀ alkenyl, aryl, alkylaryl, arylalkyl, and C₁-C₂₀ carboxylic acids;
R⁸ and R⁹ are selected from the group consisting of H, halo, C₁-C₇ alkyl, C₁-C₇ alkoxy, and combinations thereof, and
R¹⁰ is selected from the group consisting of H, C₁-C₇ alkyl, benzyl, C₁-C₇ haloalkyl, and C₁-C₇ alkoxymethyl.

9. The method of claim 1 wherein the phosphotyrosine mimetic is a compound of Formula V or a pharmaceutically acceptable salt thereof, wherein Formula V is: 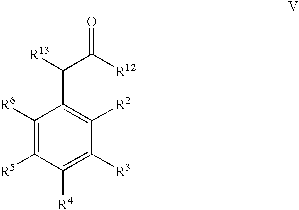
wherein
R²-R⁶ are selected from the group consisting of H, F, Cl, Br, I, NO₂, CN, OH, C₁-C₂₀ alkyl, C₁-C₂₀ alkenyl, aryl, alkylaryl, arylalkyl, C₁-C₂₀ alkoxy, R⁷OCO—, R⁷COO—, R⁷NHCO—, R⁷CONH—, peptidyl, arylpeptidyl and combinations thereof;
wherein R⁷ is selected from the group consisting of H, C₁-C₂₀ alkyl, C₁-C₂₀ alkenyl, aryl, alkylaryl, arylalkyl, and C₁-C₂₀ carboxylic acids;
R¹² is selected from the group consisting of H, C₁-C₇ alkyl, benzyl, C₁-C₇ haloalkyl, and C₁-C₇ alkoxymethyl; and
R¹³ is selected from the group consisting of H, halo, C₁-C₇ alkyl, and C₁-C₇ alkoxy.

10. The method of claim 1 wherein the phosphotyrosine mimetic is a compound of Formula VI or a pharmaceutically acceptable salt thereof, wherein Formula VI is: 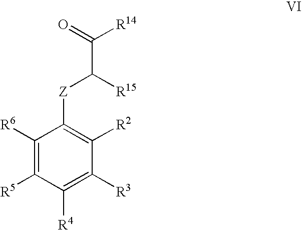
wherein
R²-R⁶ are selected from the group consisting of H, F, Cl, Br, I, NO₂, CN, OH, C₁-C₂₀ alkyl, C₁-C₂₀ alkenyl, aryl, alkylaryl, arylalkyl, C₁-C₂₀ alkoxy, R⁷OCO—, R⁷COO—, R⁷NHCO—, R⁷CONH—, peptidyl, arylpeptidyl and combinations thereof;
wherein R⁷ is selected from the group consisting of H, C₁-C₂₀ alkyl, C₁-C₂₀ alkenyl, aryl, alkylaryl, arylalkyl, and C₁-C₂₀ carboxylic acids;
R¹⁴ is selected from the group consisting of consisting of H, C₁-C₇ alkyl, benzyl, C₁-C₇ haloalkyl, and C₁-C₇ alkoxymethyl;
R¹⁵ is selected from the group consisting of H, halo, C₁-C₇ alkyl, and C₁-C₇ alkoxy; and
Z is selected from the group consisting of O, S, or NH.

11. The method of claim 1 wherein the PTP is PTP1B and the phosphotyrosine mimetic is a compound of Formula VII or a pharmaceutically acceptable salt thereof, wherein Formula VII is: 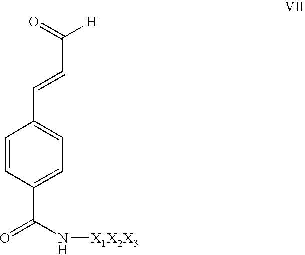
wherein X₁X₂X₃ is a tripeptide selected from the group consisting of PVL, NSV, QLL, FPS, NIY, AAF, NLG, APL, PQH, PQL, MLF, EVM, YYT, QMP, APP, NAS, GFQ, ILE, TPH, FEA, YIF, VRR, LRF, DVK, NPH, RKR, VRK, GLW, FRL, RFR, LRL, GNR, FRG, GRL, LTR, YRK, SYR RRL, GRR, SRF, LYR, YNR, RTR, TLR, GYY, LRF, NFW, TRL, YRL, RFL, FRR, RGR, RFY, NRF, GLR, DRR, NRF, RLR, RVF, ARR, FRL, DRR, TRR, RVR, RRK, FRL, QLR, RNV, NPW, FRL, FPR, GRF, and SKR.

12. The method of claim 1 wherein the PTP is PTP1B and the phosphotyrosine mimetic is a compound of Formula VIII or a pharmaceutically acceptable salt thereof, wherein Formula VIII is: 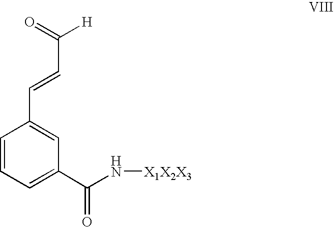
wherein X₁X₂X₃ is a tripeptide selected from the group consisting of QTQ, EGP, IHV, YNH, QVT, GVN, PVY, and PFL.

13. The method of claim 1 wherein the PTP is PTP1B and the phosphotyrosine mimetic is a compound of Formula IX or a pharmaceutically acceptable salt thereof, wherein Formula IX is: 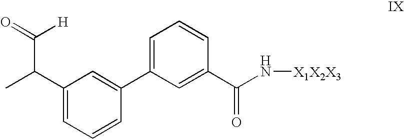
wherein X₁X₂X₃ is a tripeptide selected from the group consisting of YRY, VDW, RWR, VWA, VAR, DKA, GGA, DFL, LYM, YPY, YRL, VRM, VRF, LKW, IRF, RSF, WFL, RGR, EGA, EFP, YYR, WKV, VAW, WLR, VLL, YYR, NHY, SFW, YPL, RRA, YSP, FVG, ALG, SWA, GGA, GFN, FEY, ENV, MLM, NVS, VYM, YSL, AEN, EHL, LVY, VEM, VYT, GPT, and GTE.

14. The method of claim 1 wherein the PTP is SHP-1 and the phosphotyrosine mimetic is a compound of Formula VII or a phannaceutically acceptable salt thereof, wherein Formula VII is: 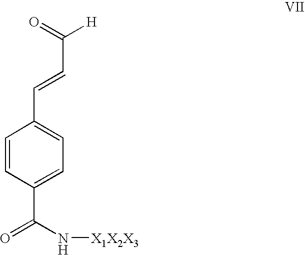
wherein X₁X₂X₃ is a tripeptide selected from the group consisting of YWY, INE, VSH, LPL, VLY, VDH, DHG, LLF, LDE, EDM, VLE, DTA, VSN, QGE, SME, FVQ, PAL, QDS, NTL, EAY, FML, IIH, VYN, NFI, VPG, GDV, and HQE.

15. The method of claim 1 wherein the PTP is SHP-1 and the phosphotyrosine mimetic is a compound of Formula VIII or a pharmaceutically acceptable salt thereof, wherein Formula VIII is: 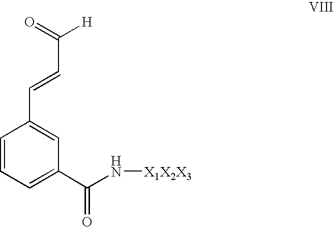
wherein X₁X₂X₃ is a tripeptide selected from the group consisting of DGL, AYV, EVA, VDL, TYG, SII, LED, QAL, QYP, VTI, and MMM.

16. The method of claim 1 wherein the PTP is SHP-1 and the phosphotyrosine mimetic is a compound of Formula IX or a pharmaceutically acceptable salt thereof, wherein Formula IX is: 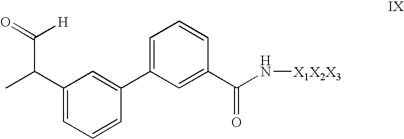
wherein X₁X₂X₃ is a tripeptide selected from the group consisting of SYF, VLF, VLV, QPF, YPA, AVA, IGP, HHA, SYP, FGA, IVT, QVS, QLV, TFH, GQY, YMI, and VVS.

17. A method of reversibly inhibiting Src homology 2 (SH2) domains, the SH2 domains having a first binding pocket comprising an arginine-containing active site and a second binding pocket comprising a second binding site, the method comprising introducing a neutral phosphotyrosine (pY) mimetic to SH2 domains, the pY mimetic comprising a reversibly binding molecule, the molecule comprising:
a. a first reactive group that forms a reversible imine or enamine adduct with the active site arginine of the first binding pocket;
b. an aryl group, wherein the aryl group provides the molecule with affinity to the active site;
c. a second reactive group that interacts with the second binding pocket; and
d. optionally, the second reactive group interacts with the reactive surface near the active site through molecular interactions, wherein the molecular interactions comprise one or more of electrostatic interactions, hydrogen-bonding interactions, hydrophobic interactions, and van der Waals interactions.

18. The method of claim 17 wherein the first reactive group is selected from aldehydes and mono-ketones.

19. The method of claim 17 wherein the phosphotyrosine mimetic is a compound of Formula I or a pharmaceutically acceptable salt thereof, wherein Formula I is: 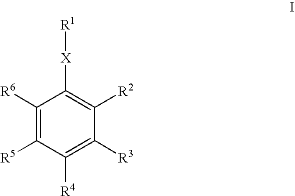
wherein
X is selected from the group consisting of C, N, O, and S;
when X is N or C, X may be further substituted with a substituent selected from the group consisting of H, halo, aryl, C₁-C₇ alkyl, and C₁-C₇ alkoxy;
R¹ is selected from the group consisting of C₁-C₇ aldehyde and C₁-C₇ ketone; and R₁ is optionally substituted at any substitutable position with H, halo, aryl, C₁-C₇ alkyl, C₁-C₇ haloalkyl, or C₁-C₇ alkoxy;
R²-R⁶ are selected from the group consisting of H, F, Cl, Br, I, NO₂, CN, OH, C₁-C₂₀ alkyl, C₁-C₂₀ alkenyl, aryl, alkylaryl, arylalkyl, C₁-C₂₀ alkoxy, R⁷0CO—, R⁷COO—, R⁷NHCO—, R⁷CONH—, peptidyl, arylpeptidyl and combinations thereof;
wherein R⁷ is selected from the group consisting of H, C₁-C₂₀ alkyl, C₁-C₂₀ alkenyl, aryl, alkylaryl, arylalkyl, and C₁-C₂₀ carboxylic acids; and
optionally, two of R²-R⁶ on adjacent C atoms may be joined to form a ring structure, wherein a fused polycyclic structure will be formed, the ring structure selected from the group consisting of aromatic, heterocyclic aromatic, saturated carbocyclic, saturated heterocyclic, partially saturated carbocyclic, and partially saturated heterocyclic.

20. The method of claim 17 wherein the phosphotyrosine mimetic is a compound of Formula II or a pharmaceutically acceptable salt thereof, wherein Formula II is: 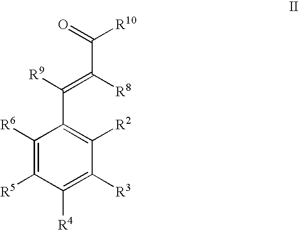
wherein
R²-R⁶ are selected from the group consisting of H, F, Cl, Br, I, NO₂, CN, OH, C₁-C₂₀ alkyl, C₁-C₂₀ alkenyl, aryl, alkylaryl, arylalkyl, C₁-C₂₀ alkoxy, R⁷OCO—, R⁷COO—, R⁷NHCO—, R⁷CONH—, peptidyl, arylpeptidyl, and combinations thereof;
wherein R⁷ is selected from the group consisting of H, C₁-C₂₀ alkyl, C₁-C₂₀ alkenyl, aryl, alkylaryl, arylalkyl, and C₁-C₂₀ carboxylic acids;
R⁸ and R⁹ are selected from the group consisting of H, halo, C₁-C₇ alkyl, and C₁-C₇ alkoxy; and
R¹⁰ is selected from the group consisting of H, C₁-C₇ alkyl, benzyl, C₁-C₇ haloalkyl, and C₁-C₇ alkoxymethyl.

21. The method of claim 17 wherein the phosphotyrosine mimetic is a compound of Formula III or a pharmaceutically acceptable salt thereof, wherein Formula III is: 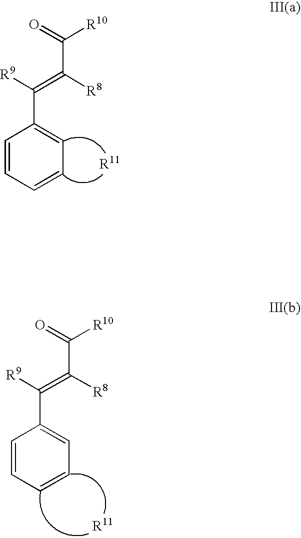
wherein
R⁸ and R⁹ are selected from the group consisting of H, halo, C₁-C₇ alkyl, and C₁-C₇ alkoxy;
R¹⁰ is selected from the group consisting of H, C₁-C₇ alkyl, benzyl, C₁-C₇ haloalkyl, and C₁-C₇ alkoxymethyl; and
R¹¹ is a fused ring selected from the group consisting of aromatic, heterocyclic aromatic, saturated carbocyclic, saturated heterocyclic, partially saturated carbocyclic, and partially saturated heterocyclic.

22. The method of claim 17 wherein the phosphotyrosine mimetic is a compound of Formula IV or a pharmaceutically acceptable salt thereof, wherein Formula IV is: 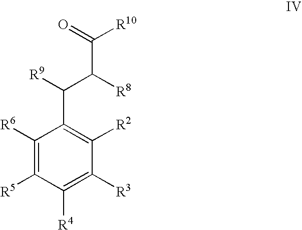
wherein
R²-R⁶ are selected from the group consisting of H, F, Cl, Br, I, NO₂, CN, OH, C₁-C₂₀ alkyl, C₁-C₂₀ alkenyl, aryl, alkylaryl, arylalkyl, C₁-C₂₀ alkoxy, R⁷OCO—, R⁷COO—, R⁷NHCO—, R⁷CONH—, peptidyl, arylpeptidyl and combinations thereof;
wherein R⁷ is selected from the group consisting of H, C₁-C₂₀ alkyl, C₁-C₂₀ alkenyl, aryl, alkylaryl, arylalkyl, and C₁-C₂₀ carboxylic acids;
R⁸ and R⁹ are selected from the group consisting of H, halo, C₁-C₇ alkyl, and C₁-C₇ alkoxy; and
R¹⁰ is selected from the group consisting of H, C₁-C₇ alkyl, benzyl, C₁-C₇ haloalkyl, and C₁-C₇ alkoxymethyl.

23. The method of claim 17 wherein the phosphotyrosine mimetic is a compound of Formula V or a pharmaceutically acceptable salt thereof, wherein Formula V is: 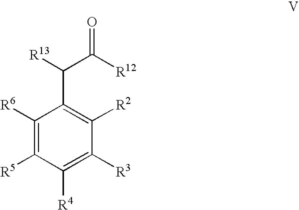
wherein
R²-R⁶ are selected from the group consisting of H, F, Cl, Br, I, NO₂, CN, OH, C₁-C₂₀ alkyl, C₁-C₂₀ alkenyl, aryl, alkylaryl, arylalkyl, C₁-C₂₀ alkoxy, R⁷OCO—, R⁷COO—, R⁷NHCO—, R⁷CONH—, peptidyl, arylpeptidyl, and combinations thereof;
wherein R⁷ is selected from the group consisting of H, C₁-C₂₀ alkyl, C₁-C₂₀ alkenyl, aryl, alkylaryl, arylalkyl, and C₁-C₂₀ carboxylic acids;
R¹² is selected from the group consisting of H, C₁-C₇ alkyl, benzyl, C₁-C₇ haloalkyl, and C₁-C₇ alkoxymethyl; and
R¹³ is selected from the group consisting of H, halo, C₁-C₇ alkyl, and C₁-C₇ alkoxy.

24. The method of claim 17 wherein the phosphotyrosine mimetic is a compound of Formula VI or a pharmaceutically acceptable salt thereof, wherein Formula VI is: 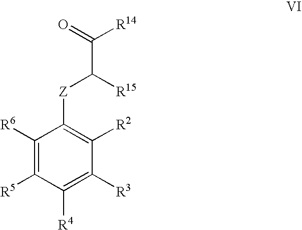
wherein
R²-R⁶ are selected from the group consisting of H, F, Cl, Br, I, NO₂, CN, OH, C₁-C₂₀ alkyl, C₁-C₂₀ alkenyl, aryl, alkylaryl, arylalkyl, C₁-C₂₀ alkoxy, R⁷OCO—, R⁷COO—, R⁷NHCO—, R⁷CONH—, peptidyl, arylpeptidyl and combinations thereof;
wherein R⁷ is selected from the group consisting of H, C₁-C₂₀ alkyl, C₁-C₂₀ alkenyl, aryl, alkylaryl, arylalkyl, and C₁-C₂₀ carboxylic acids;
R¹⁴ is selected from the group consisting of consisting of H, C₁-C₇ alkyl, benzyl, C₁-C₇ haloalkyl, and C₁-C₇ alkoxymethyl;
R¹⁵ is selected from the group consisting of H, halo, C₁-C₇ alkyl, and C₁-C₇ alkoxy; and
Z is selected from the group consisting of O, S, or NH.

25. The method of claim 17 wherein the phosphotyrosine mimetic is a compound of Formula VII or a pharmaceutically acceptable salt thereof, wherein Formula VII is: 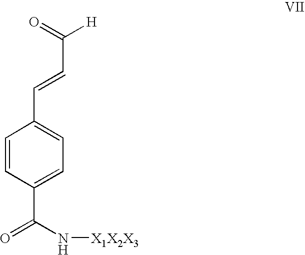
wherein X₁X₂X₃ is a tripeptide selected from the group consisting of VHL, YTR, DRN, RLQ, EEY, NDS, RGR, and RML.

26. The method of claim 17 wherein the phosphotyrosine mimetic is a compound of Formula VIII or a pharmaceutically acceptable salt thereof, wherein Formula VIII is: 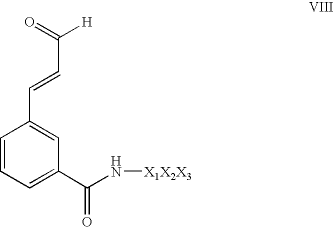
wherein X₁X₂X₃ is a tripeptide selected from the group consisting of ARL, RWL, ARN, GRT, RRV, VAR, PLL, IAH, NSR, IKL, LRR, DVR, IEF, EYR, IRF, and VKR.

27. The method of claim 17 wherein the phosphotyrosine mimetic is a compound of Formula IX or a pharmaceutically acceptable salt thereof, wherein Formula IX is: 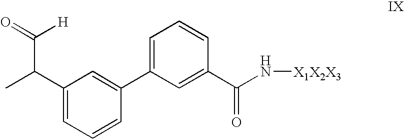
wherein X₁X₂X₃ is a tripeptide selected from the group consisting of EDY, RTH, EHV, NYP, VVT, HIH, EVF, KQI, ILR, PYY, HRM, SQY, KVR, LHF, and VHV.

28. A method of preventing or treating type II diabetes in a subject in need of such treatment, comprising administering a therapeutically effective amount of a compound of Formula II or a pharmaceutically acceptable salt thereof, wherein Formula II is: 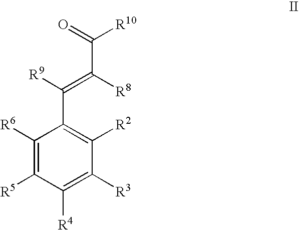
wherein
R²-R⁶ are selected from the group consisting of H, F, Cl, Br, I, NO₂, CN, OH, C₁-C₂₀ alkyl, C₁-C₂₀ alkenyl, aryl, alkylaryl, arylalkyl, C₁-C₂₀ alkoxy, R⁷0CO—, R⁷COO—, R⁷NHCO—, R⁷CONH—, peptidyl, arylpeptidyl and combinations thereof;
wherein R⁷ is selected from the group consisting of H, C₁-C₂₀ alkyl, C₁-C₂₀ alkenyl, aryl, alkylaryl, arylalkyl, and C₁-C₂₀ carboxylic acids;
R⁸ and R⁹ are selected from the group consisting of H, halo, C₁-C₇ alkyl, and C₁-C₇ alkoxy; and
R¹⁰ is selected from the group consisting of H, C₁-C₇ alkyl, benzyl, C₁-C₇ haloalkyl, and C₁-C₇ alkoxymethyl.

29. A method of probing the physiological functions of PTPs comprising adding to a PTP to be probed a neutral pY mimetic of Formula II, wherein Formula II is: 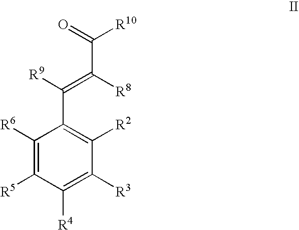
wherein
R²-R⁶ are selected from the group consisting of H, F, Cl, Br, I, NO₂, CN, OH, C₁-C₂₀ alkyl, C₁-C₂₀ alkenyl, aryl, alkylaryl, arylalkyl, C₁-C₂₀ alkoxy, R⁷0CO—, R⁷COO—, R⁷NHCO—, R⁷CONH—, peptidyl, arylpeptidyl and combinations thereof;
wherein R⁷ is selected from the group consisting of H, C₁-C₂₀ alkyl, C₁-C₂₀ alkenyl, aryl, alkylaryl, arylalkyl, and C₁-C₂₀ carboxylic acids;
R⁸ and R⁹ are selected from the group consisting of H, halo, C₁-C₇ alkyl, and C₁-C₇ alkoxy; and
R¹⁰ is selected from the group consisting of H, C₁-C₇ alkyl, benzyl, C₁-C₇ haloalkyl, and C₁-C₇ alkoxymethyl.
